(19) United States
(12) Patent Application Publication
(10) Pub. No.: US 2026/0157666 A1
(43) Pub. Date: Jun. 11, 2026

(54) OPTICAL SENSOR, OPTICAL SENSING SYSTEM, SENSING DEVICE AND METHODS OF FORMING THE SAME

(71) Applicant: Nanyang Technological University, Singapore (SG)

(72) Inventors: Yu-Cheng Chen, Singapore (SG); Ningyuan Nie, Singapore (SG)

(21) Appl. No.: 18/980,670

(22) Filed: Dec. 13, 2024

(30) Foreign Application Priority Data
Dec. 11, 2024 (SG) 10202403893X

(51) Int. Cl. A61B 5/1455; A61B 5/00

(52) U.S. Cl. CPC A61B 5/1455; A61B 5/6833; A61B 2562/0233; A61B 2562/12

(57) ABSTRACT

An optical sensor for detecting an analyte. The optical sensor may include a hydrogel film, and one or more cholesteric liquid crystal (CLC) droplets mixed with the hydrogel film. Each of the one or more cholesteric liquid crystal droplets may include a plurality of liquid crystal molecules. The optical sensor may also include modification molecules attached to the one or more cholesteric liquid crystal droplets. The one or more cholesteric liquid crystal droplets may be doped with a photoluminescent dye, may be configured to generate an emission beam in response to a pump laser beam incident onto the one or more cholesteric liquid crystals droplets, and may be configured as whispering gallery mode (WGM) resonators such that a wavelength of the emission beam is shifted in response to the modification molecules coming in contact with a chemical derived from the analyte, thereby detecting the analyte.

### CROSS-REFERENCE TO RELATED APPLICATION

This application claims the benefit of priority of Singapore Application No. 10202403893X filed Dec. 11, 2024, the contents of it being hereby incorporated by reference in its entirety for all purposes.

### TECHNICAL FIELD

Various embodiments of this disclosure may relate to an optical sensor. Various embodiments of this disclosure may relate to an optical sensing system. Various embodiments may relate to a wearable analyte sensing device. Various embodiments may relate to a method of forming an optical sensor for detecting an analyte. Various embodiments may relate to a method of forming an optical sensing system. Various embodiments may relate to a method of forming a wearable analyte sensing device.

### BACKGROUND

Flexible devices have received tremendous attention in the past decade owing to their lightweightness, bendability, stretchability, and ease of integration with human interfaces. By bridging flexible materials with photonics, flexible photonics offered the possibility to realize flexible light sources, flexible displays, solar cells, flexible photodetectors, anticounterfeiting labels, and wearable sensors. Recently, wearable photonic sensors have been extensively explored as an alternative to state-of-the-art wearable sensors owing to their resistance to electromagnetic radiation and environmental changes. Optical wearables are also known for their potential capability to perform remote sensing and detection of multiple parameters at the same time. To date, optical wearable sensors have been developed to detect humidity, physical motions, respiration, heart rate, and temperature. Various microscale and nanoscale photonic resonators have been incorporated into flexible materials to improve the sensitivity of wearable optical sensors, including plasmonics, Bragg grating, fiber, and whispering gallery mode microresonators. For instance, a flexible organic microlaser array has been developed which can detect the gesture of human fingers. A flexible bandgap nanolaser with a semiconductor slab embedded in polymers and yielding an optical strain sensitivity for mechanical detection has also been demonstrated. Additionally, there are various types of flexible lasers fabricated from plasmonic fibers and microrings to detect environmental changes on the surface of the human body.

Direct sensing of biochemicals released in the human body can provide more clinical-relevant bioinformation. As a matter of fact, human sweat contains a plethora of biomarkers which provide key physiological information related to human function and metabolism. Compared with blood testing, sweat testing offers the advantages of noninvasiveness, portability, and persistence. Hence, the analysis and detection of biomarkers in sweat can assist in the prevention, diagnosis, and especially monitoring of chronic diseases. Previous studies have investigated the possibility of using surface-enhanced Raman scattering, photonic crystal-based structural color, and polarized microscope for sweat sensing. Despite the rapid advancement in wearable optical sensors, one of the greatest challenges is to detect multiple biochemicals on a single device, i.e., the device is capable of multiplexed detection or multifunctionality.

### SUMMARY

Various embodiments may relate to an optical sensor for detecting an analyte. The optical sensor may include a hydrogel film. The optical sensor may also include one or more cholesteric liquid crystal (CLC) droplets mixed with the hydrogel film. Each of the one or more cholesteric liquid crystal (CLC) droplets may include a plurality of liquid crystal molecules. The optical sensor may also include modification molecules attached to the one or more cholesteric liquid crystal (CLC) droplets. The one or more cholesteric liquid crystal (CLC) droplets may be doped with a photoluminescent dye. The one or more cholesteric liquid crystals (CLC) droplets may be configured to generate an emission beam in response to a pump laser beam incident onto the one or more cholesteric liquid crystals (CLC) droplets. The one or more cholesteric liquid crystal (CLC) droplets may be configured as whispering gallery mode (WGM) resonators such that a wavelength of the emission beam is shifted in response to the modification molecules coming in contact with a chemical derived from the analyte, thereby detecting the analyte.

Various embodiments may relate to an optical sensing system. The optical sensing system may include one or more optical sensors as described herein. The optical sensing system may also include a pump lasing sub-system configured to provide the pump laser beam. The optical sensing system may further include a detection sub-system configured to detect the emission beam.

Various embodiments may relate to a wearable analyte sensing device. The wearable analyte sensing device may include one or more optical sensors as described herein. The wearable analyte sensing device may also include a substrate holding the one or more optical sensors. The wearable analyte sensing device may further include an adhesion layer for adhering the wearable analyte sensing device to an user. The one or more optical sensors may be between the substrate and the adhesion layer.

Various embodiments may relate to a method of forming an optical sensor for detecting an analyte. The method may include forming one or more cholesteric liquid crystal (CLC) droplets, each of the one or more cholesteric liquid crystal (CLC) droplets comprising a plurality of liquid crystal molecules. The method may also include attaching modification molecules to the one or more cholesteric liquid crystal (CLC) droplets. The method may further include doping the one or more cholesteric liquid crystal (CLC) droplets with a photoluminescent dye. The method may additionally include mixing the one or more cholesteric liquid crystal (CLC) droplets with the hydrogel film. The one or more cholesteric liquid crystals (CLC) droplets may be configured to generate an emission beam in response to a pump laser beam incident onto the one or more cholesteric liquid crystals (CLC) droplets. The one or more cholesteric liquid crystal (CLC) droplets may also be configured as whispering gallery mode (WGM) resonators such that a wavelength of the emission beam is shifted in response to the modification molecules coming in contact with a chemical derived from the analyte, thereby detecting the analyte.

Various embodiments may relate to a method of forming an optical sensing system. The method may include forming or providing one or more optical sensors as described herein. The method may also include providing a pump lasing sub-system configured to provide the pump laser beam. The method may further include providing a detection sub-system configured to detect the emission beam.

Various embodiments may relate to a method of forming a wearable analyte sensing device. The method may include forming one or more optical sensors as described herein. The method may also include forming a substrate to hold the one or more optical sensors. The method may further include forming an adhesion layer for adhering the wearable analyte sensing device to an user. The one or more optical sensors may be between the substrate and the adhesion layer.

### BRIEF DESCRIPTION OF THE DRAWINGS

In the drawings, like reference characters generally refer to the same parts throughout the different views. The drawings are not necessarily drawn to scale, emphasis instead generally being placed upon illustrating the principles of various embodiments. In the following description, various embodiments of the invention are described with reference to the following drawings.

FIG. 1 shows a schematic of an optical sensor for detecting an analyte according to various embodiments.

FIG. 2 shows a schematic of an optical sensing system according to various embodiments.

FIG. 3 shows a schematic of a wearable analyte sensing device according to various embodiments.

FIG. 4 shows a schematic illustrating a method of forming an optical sensor for detecting an analyte according to various embodiments.

FIG. 5 shows a schematic illustrating a method of forming an optical sensing system according to various embodiments.

FIG. 6 shows a schematic illustrating a method of forming a wearable analyte sensing device according to various embodiments.

FIG. 7A shows a schematic of the optical sensor being used for sweat sensing according to various embodiments.

FIG. 7B shows a schematic illustrating the sensing mechanism of the modified cholesteric liquid crystal (CLC) droplets for generating whispering galley modes according to various embodiments.

FIG. 7C shows captured photoluminescence images of the luminescent modified cholesteric liquid crystal (CLC) droplets doped with different dyes and embedded in the hydrogel film according to various embodiments.

FIG. 7D shows the molecular structures of (a) 4′-Pentyl-4-biphenylcarbonitrile (5CB), (b) (S)-4-Cyano-4′-(2-methylbutyl) biphenyl, (c) 1,3,5,7-tetramethyl-8-phenyl-4,4-difluoroboradiazaindacene (Bodipy-1), (d) 2,8-diethyl-1,3,5,7-tetramethyl-9-phenylbipyrromethene difluoroborate (Bodipy-2), and (e) Nile red according to various embodiments.

FIG. 7E shows a three-dimensional (3D) plot of intensity as a function of wavelength (in nanometer or nm) and dye type illustrating the absorption/photoluminescence properties of the dyes according to various embodiments.

FIG. 8A shows a plot of intensity (in arbitrary units or a.u.) as a function of wavelength (in nanometers or nm) illustrating the lasing spectra of modified cholesteric liquid crystal (CLC) microdroplets according to various embodiments in poly(vinyl alcohol) (PVA) solution under different pump energy densities.

FIG. 8B shows a plot of intensity (in arbitrary units or a.u.) as a function of wavelength (in nanometers or nm) illustrating the lasing spectra of modified cholesteric liquid crystal (CLC) microdroplets according to various embodiments in polyacrylamide (PAAm) under different pump energy densities.

FIG. 8C shows a schematic of the optical sensing system or setup according to various embodiments.

FIG. 8D shows (above) a schematic illustrating fabrication of cholesteric liquid crystal (CLC) microdroplets in the polyacrylamide (PAAm) according to various embodiments in a mold of 10 mm×15 mm×1 mm; and (below) a schematic illustrating fabrication of cholesteric liquid crystal (CLC) microdroplets in the polyacrylamide (PAAm) according to various embodiments in circular molds of diameters of 8 mm.

FIG. 8E shows a plot of normalized intensity (in arbitrary units or a.u.) as a function of pump energy density (in micro-Joules per square milli-meter or mJ/mm²) illustrating the lasing threshold of dye-doped cholesteric liquid crystal (CLC) microdroplets in poly(vinyl alcohol) (PVA) and polyacrylamide (PAAm) according to various embodiments.

FIG. 8F shows a plot of normalized intensity (in arbitrary units or a.u.) as a function of pump energy density (in micro-Joules per square milli-meter or mJ/mm²) illustrating the lasing thresholds of cholesteric liquid crystal (CLC) in polyacrylamide (PAAm) with different dyes (Bodipy-1, Bodipy-2, and Nile Red) according to various embodiments, with insets showing bright whispering gallery mode (WGM) resonators lasing emissions observed at the outer boundary of the microdroplets.

FIG. 8G shows (above) a schematic of the bending test for the polyacrylamide (PAAm) hydrogel film laser according to various embodiments; and (below) the bending stress distribution of the polyacrylamide (PAAm) hydrogel film according to various embodiments.

FIG. 8H shows (above) a schematic of the incubator used for the temperature stability test of the polyacrylamide (PAAm) hydrogel film laser according to various embodiments; and (below) a schematic of the temperature stability test conducted on the polyacrylamide (PAAm) hydrogel film laser according to various embodiments.

FIG. 8I shows a plot of normalized intensity (in arbitrary units or a.u.) as a function wavelength (in nanometers or nm) illustrating the lasing spectra of Nile Red-doped cholesteric liquid crystal (CLC) microdroplets in polyacrylamide (PAAm) with different bending angles (0° to) 120°, with the insets showing morphology of the cholesteric liquid crystal (CLC) microdroplets at different bending angles.

FIG. 8J shows a plot of normalized intensity (in arbitrary units or a.u.) as a function wavelength (in nanometers or nm) illustrating the lasing spectra of Bodipy-1-doped cholesteric liquid crystal (CLC) microdroplets in polyacrylamide (PAAm) under different environmental temperatures, from 27° C. to 40° C.

FIG. 9A shows the modification of the cholesteric liquid crystal (CLC) microdroplets for (a) lactate sensing, (b) glucose sensing and (c) urea sensing according to various embodiments.

FIG. 9B shows a schematic illustrating the working principle of the modified cholesteric liquid crystal (CLC) microdroplet for lactate sensing according to various embodiments.

FIG. 9C shows whispering galley mode (WGM) polarization schematics in liquid crystal (LC) droplets illustrating (a) electric filed direction for transverse magnetic (TM) modes according to various embodiments; and (b) the birefringence of a single liquid crystal (LC) molecule.

FIG. 9D shows plots of normalized intensity (in arbitrary units or a,u.) as a function of wavelength (in nanometer or nm) illustrating lasing spectra of the modified cholesteric liquid crystal (CLC) microdroplets in hydrogel under different lactate concentrations according to various embodiments.

FIG. 9E shows a plot of wavelength shift (in nanometers or nm) as a function of time (in minutes or min) illustrating the wavelength shifts with increased lactate concentration after 2 minutes, 4 minutes, and 6 minutes according to various embodiments.

FIG. 9F shows a plot of wavelength shift (in nanometers or nm) as a function of concentration of lactate (in milli-molars or mM) illustrating the wavelength shifts with increased lactate concentration under a fixed observation time (6 minutes) according to various embodiments.

FIG. 10A shows a schematic illustrating the working principle of the modified cholesteric liquid crystal (CLC) microdroplet for glucose and urea sensing according to various embodiments.

FIG. 10B shows (above) a plot of wavelength shift (in nanometers or nm) as a function of time (in minutes or min) illustrating the lasing spectra of the modified cholesteric liquid crystal (CLC) microdroplet in the hydrogel under different glucose concentrations according to various embodiments; and (below) a plot of wavelength shift (in nanometers or nm) as a function of glucose concentration (in micro-molars or μM) illustrating the wavelength shift of the lasing spectra of the modified cholesteric liquid crystal (CLC) microdroplet in the hydrogel under different glucose concentrations according to various embodiments.

FIG. 10C shows (above) a plot of wavelength shift (in nanometers or nm) as a function of time (in minutes or min) illustrating the lasing spectra of the modified cholesteric liquid crystal (CLC) microdroplet in the hydrogel under different urea concentrations according to various embodiments; and (below) a plot of wavelength shift (in nanometers or nm) as a function of urea concentration (in micro-molars or μM) illustrating the wavelength shift of the lasing spectra of the modified cholesteric liquid crystal (CLC) microdroplet in the hydrogel under different urea concentrations according to various embodiments.

FIG. 10D shows plots of wavelength shift (in nanometers or nm) as a function of different solutions/mixtures illustrating selectivity of the optical sensor according to various embodiments. Error bars are obtained based on quintuplicate measurements.

FIG. 11A shows a schematic illustrating the wearable sensing device according to various embodiments adhered to human skin for direct sweat sensing.

FIG. 11B shows (above) a plot of wavelength shift (in nanometers or nm) as a function of time (in hours or h) illustrating the wavelength shift of emission light emitted by the wearable sensing device according to various embodiments before and after protein intake for 2 hours; and (below) plots of normalized intensity (in arbitrary units or a.u.) as a function of wavelength (in nanometers or nm) illustrating the lasing spectra shown by the wearable sensing device according to various embodiments before and after protein intake for 2 hours.

FIG. 11C shows (above) a plot of wavelength shift (in nanometers or nm) as a function of time (in hours or h) illustrating the wavelength shift of emission light emitted by the wearable sensing device according to various embodiments before and after glucose intake for 2 hours; and (below) plots of normalized intensity (in arbitrary units or a.u.) as a function of wavelength (in nanometers or nm) illustrating the lasing spectra shown by the wearable sensing device according to various embodiments before and after glucose intake for 2 hours.

FIG. 11D shows (above) a plot of wavelength shift (in nanometers or nm) as a function of activity illustrating the wavelength shift of emission light emitted by the wearable sensing device according to various embodiments after jogging for 10 minutes and after high-intensity interval training (HIIT) for 10 minutes; and (below) plots of normalized intensity (in arbitrary units or a.u.) as a function of wavelength (in nanometers or nm) illustrating the lasing spectra shown by the wearable sensing device according to various embodiments after jogging for 10 minutes and after high-intensity interval training (HIIT) for 10 minutes.

### DESCRIPTION

The following detailed description refers to the accompanying drawings that show, by way of illustration, specific details and embodiments in which the invention may be practiced. These embodiments are described in sufficient detail to enable those skilled in the art to practice the invention. Other embodiments may be utilized and structural, logical, and electrical changes may be made without departing from the scope of the invention. The various embodiments are not necessarily mutually exclusive, as some embodiments can be combined with one or more other embodiments to form new embodiments.

Features that are described in the context of an embodiment may correspondingly be applicable to the same or similar features in the other embodiments. Features that are described in the context of an embodiment may correspondingly be applicable to the other embodiments, even if not explicitly described in these other embodiments. Furthermore, additions and/or combinations and/or alternatives as described for a feature in the context of an embodiment may correspondingly be applicable to the same or similar feature in the other embodiments.

In the context of various embodiments, the articles “a”, “an” and “the” as used with regard to a feature or element include a reference to one or more of the features or elements.

In the context of various embodiments, the term “about” or “approximately” as applied to a numeric value encompasses the exact value and a reasonable variance, e.g., within 10% of the specified value.

As used herein, the term “and/or” includes any and all combinations of one or more of the associated listed items.

By “comprising” it is meant including, but not limited to, whatever follows the word “comprising”. Thus, use of the term “comprising” indicates that the listed elements are required or mandatory, but that other elements are optional and may or may not be present.

By “consisting of” it is meant including, and limited to, whatever follows the phrase “consisting of”. Thus, the phrase “consisting of” indicates that the listed elements are required or mandatory, and that no other elements may be present.

Embodiments described in the context of one of the optical sensors/optical sensing systems/wearable analyte sensing devices are analogously valid for the other optical sensors/optical sensing systems/wearable analyte sensing devices. Similarly, embodiments described in the context of a method are analogously valid for an optical sensor/optical sensing system/wearable analyte sensing device, and vice versa.

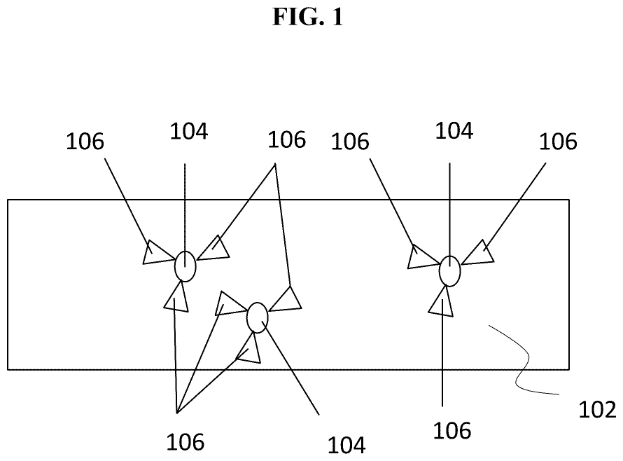

FIG. 1 shows a schematic of an optical sensor for detecting an analyte according to various embodiments. The optical sensor may include a hydrogel film 102. The optical sensor may also include one or more cholesteric liquid crystal (CLC) droplets 104 mixed with the hydrogel film 102. Each of the one or more cholesteric liquid crystal (CLC) droplets 104 may include a plurality of liquid crystal molecules. The optical sensor may also include modification molecules 106 attached to the one or more cholesteric liquid crystal (CLC) droplets 104. The one or more cholesteric liquid crystal (CLC) droplets 104 may be doped with a photoluminescent dye. The one or more cholesteric liquid crystals (CLC) droplets 104 may be configured to generate an emission beam in response to a pump laser beam incident onto the one or more cholesteric liquid crystals (CLC) droplets 104. The one or more cholesteric liquid crystal (CLC) droplets 104 may be configured as whispering gallery mode (WGM) resonators such that a wavelength of the emission beam is shifted in response to the modification molecules 106 coming in contact with a chemical derived from the analyte, thereby detecting the analyte.

In other words, various embodiments may relate to a hydrogel film 102 with one or more cholesteric liquid crystal (CLC) droplets 104 dispersed in the hydrogel film 102. Each droplet may be doped with a photoluminescent dye and may be attached to one or more modification molecules 106. Upon illumination with a pump laser beam, the droplets 104 may emit an emission beam with a wavelength shift that is dependent on a chemical that comes into contact the modification molecules 106. The chemical may be derived from a specific analyte. The analyte may therefore be determined or detected via the wavelength shift of the emission beam.

For avoidance of doubt, FIG. 1 is intended to illustrate some features of an optical sensor according to various embodiments, and is not intended to limit the number, shapes, sizes/dimensions, orientation, arrangement etc. of the various embodiments. For instance, while FIG. 1 illustrates three droplets 104, and three modification molecules 106 attached to each droplet 104, various embodiments may have any suitable number (e.g., 1, 2 . . . 100, 1000 etc.) of droplets 104, and any suitable number of modification molecules 106 (e.g., 1, 2, 3 . . . etc.) attached to each droplet 104. While FIG. 1 shows the same number of modification molecules 106 (i.e. three) attached to each droplet 104, various embodiments may have different number of modification molecules 106 attached to each droplet 104. Additionally, while FIG. 1 depicts circular droplets 104 being circular, the droplets 104 may be of any suitable shape, e.g., teardrop shaped, oval etc. Different droplets 104 may have the same shape and/or size or may be of different shapes and/or sizes. Additionally, while FIG. 1 depicts the modification molecules 106 to be of triangular shapes, such depictions are for illustration purposes and modification molecules 106 can be of any suitable shape or shapes.

The optical sensor may also be referred to as a wearable thin film laser or a hydrogel film laser.

In various embodiments, the hydrogel film 102 may include an enzyme configured to react with the analyte to form the chemical.

In various embodiments, the shift in the wavelength of the emission beam may be due to a change in orientation of the plurality of liquid crystal molecules as a result of a reaction between the chemical and the modification molecules 106. Depending on the chemical type and/or concentration that reacts with the modification molecules 106, emission beams with different wavelengths may be emitted. The concentration of the chemical formed may be dependent on the concentration of the analyte that is absorbed by the hydrogel film 102. In various embodiments, the type and/or concentration of the analyte may be determined based on the shift in the wavelength of the emission beam.

In various embodiments, the modification molecules 106 may be thiol molecules or carboxylic acid molecules.

In various embodiments, the photoluminescent dye may be selected from a group consisting of 1,3,5,7-tetramethyl-8-phenyl-4,4-difluoroboradiazaindacene (Bodipy-1), Nile Red, and 2,8-diethyl-1,3,5,7-tetramethyl-9-phenylbipyrromethene difluoroborate (Bodipy-2). The wavelength of the emission beam may be dependent on the photoluminescent dye.

In various embodiments, the hydrogel film 102 may include any suitable hydrogel material, e.g., polyacrylamide (PAAm).

Whispering gallery mode resonators may be structures capable of trapping light in paths around peripheries of the structures (i.e., similar to those taken by sound waves traveling from one person to another in a circular room or whispering gallery). The one or more CLC droplets 104 being configured as whispering gallery mode resonators may refer to the one or more CLC droplets 104 being shaped or configured to trap light around an edge region of each of the one or more CLC droplets 104. The pump laser beam that is provided to the optical sensor may be absorbed by the photoluminescent dye which may generate a light after absorbing the pump laser beam. The light generated by the photoluminescent dye may have a wavelength different from the wavelength of the optical pump light. The wavelength of the light generated by the photoluminescent dye may be dependent on a molecular structure of the photoluminescent dye. The light that is generated by the photoluminescent dye may be trapped in the edge region of a resonator until the light reaches sufficient intensity and the resonator emits the emission beam. Accordingly, the wavelength of the emission beam generated by the resonator may be dependent on a refractive index of the CLC droplet 104 and a size of the CLC droplet 104, in addition to the molecular structure of photoluminescent dye. However, once an optical sensor is fabricated, the size of the CLC droplets 104 and the molecular structure of the photoluminescent dye in the optical sensor may be fixed. However, the refractive index of the CLC droplets 104 may be variable owing to the birefringence of liquid crystal molecules, and may be dependent on the rotation of the liquid crystal molecules in the CLC droplets 104. The rotation of the liquid crystal molecules may be in turn be dependent on the chemical that comes into contact the modification molecules 106, as mentioned above. Consequently, the wavelength of the emission beam may be shifted depending on the chemical that comes into contact the modification molecules 106.

In various embodiments, the analyte may be lactate, glucose or urea. In various other embodiments, the analyte may be any other suitable substances, e.g., biomolecules such drugs or pathological chemicals.

In the case of lactate, the lactate that is absorbed by the hydrogel film 102 may come into contact with the enzyme lactate oxidase (LOx) in the hydrogel film 102, and may be oxidized to generate chemicals pyruvate and hydrogen peroxide (H₂O₂). The chemicals generated may oxidize the modification molecules 106 (which may be thiol molecules such as 1-dodecanethiol). The oxidation of the molecules 106 may lead to rotation of the liquid crystal molecules, thereby leading to a change in the refractive index of the liquid crystal (CLC) droplets 104, and a change in wavelength (blue shift) of the emission beam generated in response to or upon incidence of the pump laser beam.

In the case of glucose, the glucose that is absorbed by the hydrogel film 102 may come into contact with the enzyme glucose oxidase (GOx) in the hydrogel film 102, and may be oxidized to generate a chemical hydrogen peroxide (H₂O₂). The chemical generated may oxidize the modification molecules 106 (which may be thiol molecules such as 1-dodecanethiol). Similarly, the oxidation of the molecules 106 may lead to rotation of the liquid crystal molecules, thereby leading to a change in the refractive index of the liquid crystal (CLC) droplets 104, and a change in wavelength (blue shift) of the emission beam generated in response to or upon incidence of the pump laser beam.

In the case of urea, the urea that is absorbed by the hydrogel film 102 may come into contact with the enzyme urease in the hydrogel film 102, and be oxidized to generate a chemical ammonia (NH₃). The chemical generated may oxidize the modification molecules 106 (which may be carboxylic acid molecules). The oxidation of the molecules 106 may lead to reverse rotation of the liquid crystal molecules, thereby leading to a change in the refractive index of the liquid crystal (CLC) droplets 104, and a change in wavelength (red shift) of the emission beam generated in response to or upon incidence of the pump laser beam.

Various embodiments may be used to determine or detect other types of analyte. The enzyme in the hydrogel film 102 and/or the modification molecules 106 used may be dependent on the type of analyte to be detected or determined.

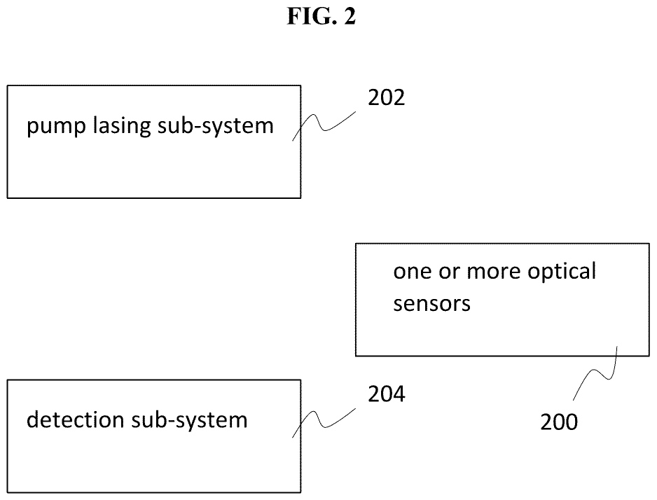

FIG. 2 shows a schematic of an optical sensing system according to various embodiments. The optical sensing system may include one or more optical sensors 200 as described herein. The optical sensing system may also include a pump lasing sub-system 202 configured to provide the pump laser beam. The optical sensing system may further include a detection sub-system 204 configured to detect the emission beam.

In other words, various embodiments may relate to an optical sensing system including a pump lasing sub-system 202 for providing the pump laser beam, and a detection sub-system 204 for detecting the emission beam.

For avoidance of doubt, FIG. 2 is intended to illustrate some features of the optical sensing system according to various embodiments, and is not intended to limit the arrangement, shapes, sizes, orientation etc. of the various features.

In various embodiments, the detection sub-system 204 may include a charge-coupled device (CCD) and a spectrometer.

In various embodiments, the pump lasing sub-system 202 may be or may include a pump laser source.

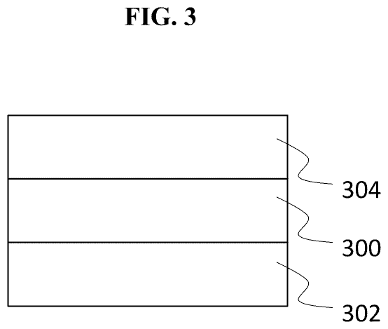

FIG. 3 shows a schematic of a wearable analyte sensing device according to various embodiments. The wearable analyte sensing device may include one or more optical sensors 300 as described herein. The wearable analyte sensing device may also include a substrate 302 holding the one or more optical sensors 300. The wearable analyte sensing device may further include an adhesion layer 304 for adhering the wearable analyte sensing device to an user. The one or more optical sensors 300 may be between the substrate 302 and the adhesion layer 304.

For avoidance of doubt, FIG. 3 is intended to illustrate some features of the wearable sensing device according to various embodiments, and is not intended to limit the shapes, sizes, dimensions, orientation etc. of the various features.

The wearable analyte sensing device may be referred to as an emitting plaster (or bandage).

In various embodiments, the adhesion layer 304 may be a medical tape.

In various embodiments, the substrate 302 may include any suitable material, such as polydimethylsiloxane (PDMS). In various embodiments, the substrate 302 may be an adhesive bandage.

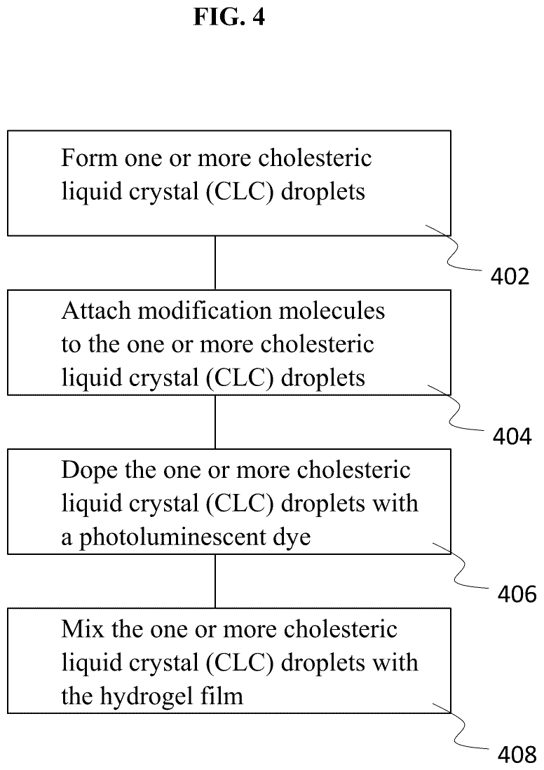

FIG. 4 shows a schematic illustrating a method of forming an optical sensor for detecting an analyte according to various embodiments. The method may include, in 402, forming one or more cholesteric liquid crystal (CLC) droplets, each of the one or more cholesteric liquid crystal (CLC) droplets comprising a plurality of liquid crystal molecules. The method may also include, in 404, attaching modification molecules to the one or more cholesteric liquid crystal (CLC) droplets. The method may further include, in 406, doping the one or more cholesteric liquid crystal (CLC) droplets with a photoluminescent dye. The method may additionally include, in 408, mixing the one or more cholesteric liquid crystal (CLC) droplets with the hydrogel film. The one or more cholesteric liquid crystals (CLC) droplets may be configured to generate an emission beam in response to a pump laser beam incident onto the one or more cholesteric liquid crystals (CLC) droplets. The one or more cholesteric liquid crystal (CLC) droplets may also be configured as whispering gallery mode (WGM) resonators such that a wavelength of the emission beam is shifted in response to the modification molecules coming in contact with a chemical derived from the analyte, thereby detecting the analyte.

For avoidance of doubt, FIG. 4 seeks to illustrate some steps of a method of forming an optical sensor according to various embodiments, and is not intended to limit the sequence of the various steps.

In various embodiments, the one or more cholesteric liquid crystal (CLC) droplets may first be formed (i.e., step 402), followed by attaching of the modification molecules and doping of the CLC droplets (i.e., steps 404, 406).

In various embodiments, the one or more cholesteric liquid crystal (CLC) droplets may be doped with the photoluminescent dye and attached to the modification molecules (i.e., steps 404, 406), before mixing the one or more cholesteric liquid crystal (CLC) droplets doped with the photoluminescent dye and functionalized with the modification molecules with the hydrogel film (i.e., step 408).

In various embodiments, step 404 may occur before, after or at the same time as step 406. For instance, a cholesteric liquid crystal (CLC) mixture may be mixed with the photoluminescent dye and the modification molecules to form the one or more cholesteric liquid crystal (CLC) droplets doped with the photoluminescent dye and functionalized with the modification molecules.

In various embodiments, the modification molecules may be thiol molecules or carboxylic acid molecules. In various embodiments, the photoluminescent dye may be selected from a group consisting of Bodipy-1, Bodipy-2, and Nile Red.

In various embodiments, the hydrogel film may include an enzyme configured to react with the analyte to form the chemical.

In various embodiments, the hydrogel film may be formed by mixing a hydrogel precursor solution with a photoinitiator to form a resultant mixture; and providing ultraviolet (UV) light to the resultant mixture.

In various embodiments, the hydrogel film may include any suitable hydrogel material, e.g., polyacrylamide (PAAm).

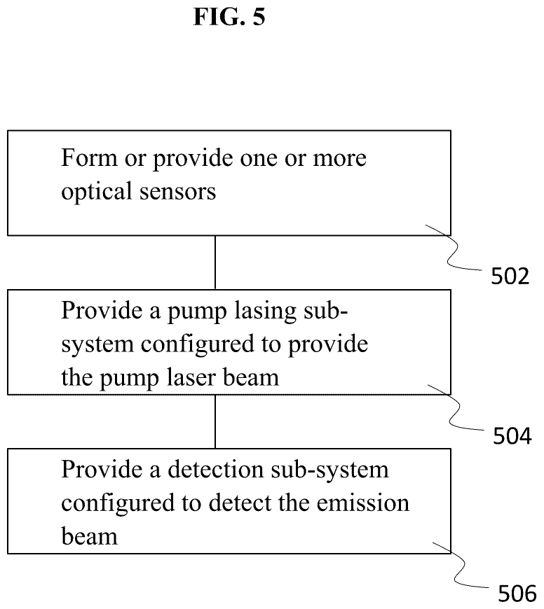

FIG. 5 shows a schematic illustrating a method of forming an optical sensing system according to various embodiments. The method may include, in 502, forming or providing one or more optical sensors as described herein. The method may also include, in 504, providing a pump lasing sub-system configured to provide the pump laser beam. The method may further include, in 506, providing a detection sub-system configured to detect the emission beam.

For avoidance of doubt, FIG. 5 seeks to illustrate some steps of a method of forming an optical sensing system according to various embodiments, and is not intended to limit the sequence of the various steps. For instance, step 502 may occur before, after or at the same time as step 504.

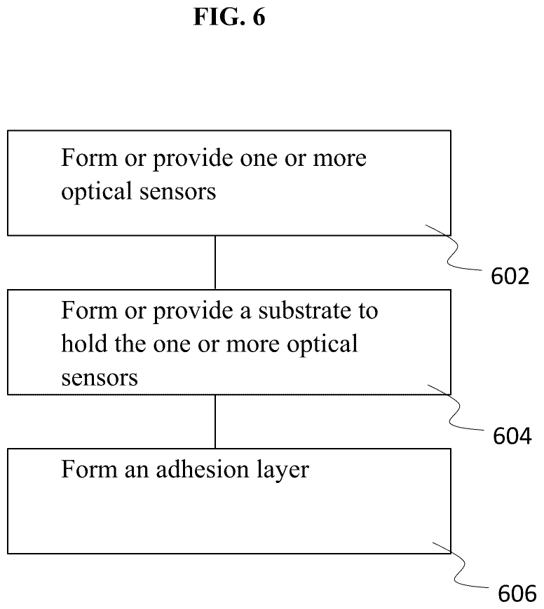

FIG. 6 shows a schematic illustrating a method of forming a wearable analyte sensing device according to various embodiments. The method may include, in 602, forming or providing one or more optical sensors as described herein. The method may also include, in 604, forming or providing a substrate to hold the one or more optical sensors. The method may further include, in 606, forming an adhesion layer for adhering the wearable analyte sensing device to an user. The one or more optical sensors may be between the substrate and the adhesion layer.

For avoidance of doubt, FIG. 6 seeks to illustrate some steps of a method of forming a wearable analyte sensing device according to various embodiments, and is not intended to limit the sequence of the various steps. For instance, step 602 may occur before, after or at the same time as step 604. In various embodiments, step 606 may occur after steps 602 and 604.

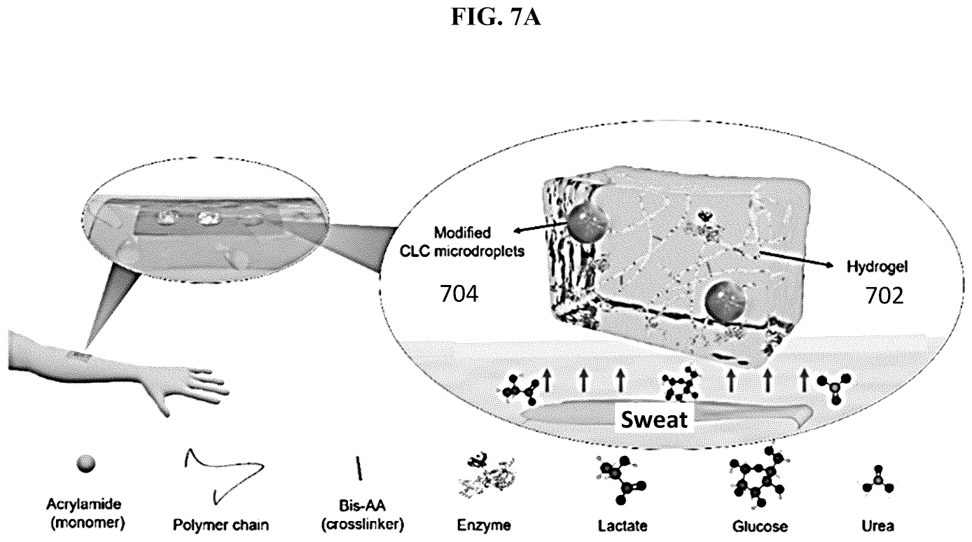

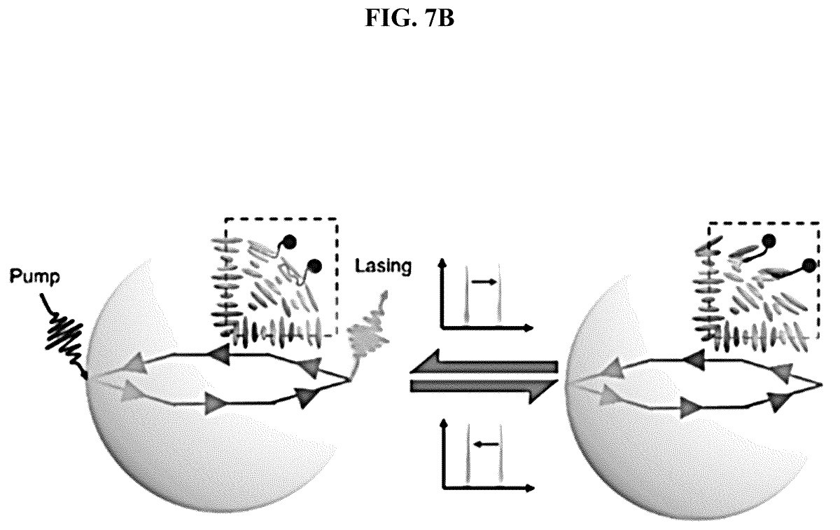

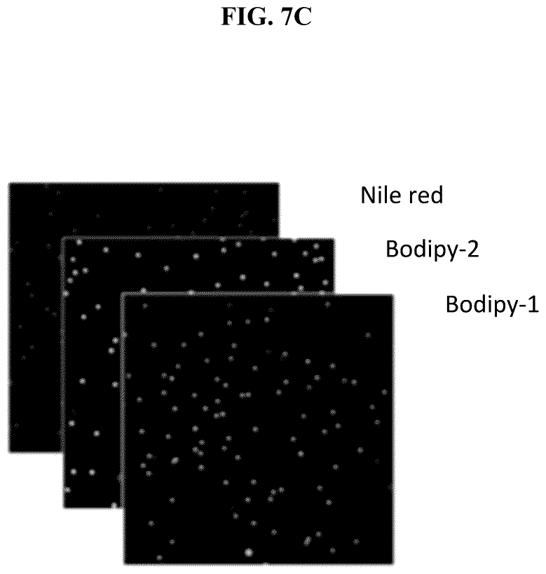

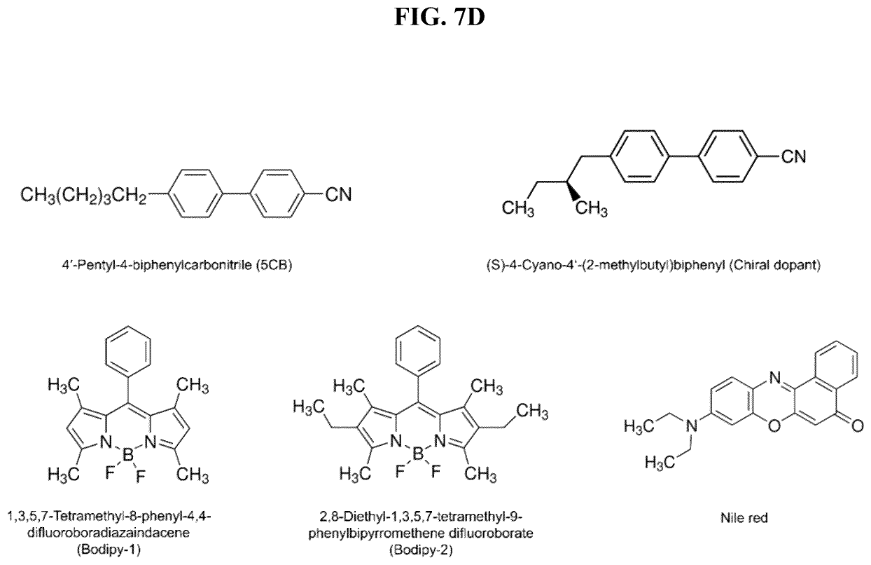

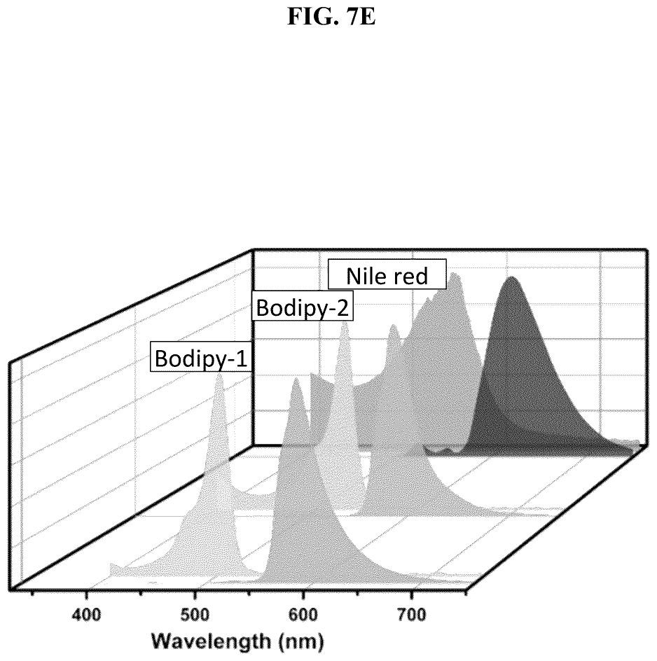

Stimulated emissions from micro- to nanoscale lasers may offer unique advantages in terms of signal amplification and narrow line width. Strong light interactions between optical microcavities and biomolecules would therefore lead to distinctive lasing signals for sensing. In particular, droplet-based microlasers are promising candidates for their compact size, ease of fabrication, biocompatibility, and high-quality factor for sensing. Microdroplet lasers made from various types of materials have been demonstrated for sensing in solutions and body fluids; however, they have not been deployed directly on human or physiological sensing applications before. To obtain an active microlaser with biochemical sensing functions, a wearable thin film laser may be formed by encapsulating cholesteric liquid crystal (CLC) droplets in a flexible hydrogel thin film. FIG. 7A shows a schematic of the optical sensor being used for sweat sensing according to various embodiments. The optical sensor may include a hydrogel film 702 including modified cholesteric liquid crystal (CLC) droplets 704. Each single CLC microdroplet may serve as a whispering gallery mode (WGM) resonator, and the lasing wavelength may be determined by the gain (fluorescence dye) doped within liquid crystal molecules. The three-dimensional cross-linked hydrophilic polymer (i.e., of the hydrogel film 702) may serve as the adhesive layer to allow small molecules (i.e., analytes or metabolites) to diffuse from human tissue to the surface of droplet laser resonators. Ascribable to the high-quality factor of the whispering gallery mode (WGM) resonator, subtle changes in the liquid crystal droplets may be amplified, resulting in a wavelength shift in the laser emission spectra, which can then be applied for sensing and monitoring metabolites.

To achieve the desired sensing functionality for lactate, glucose, and urea, CLC microdroplets doped with different photoluminescent dyes may be modified with specific molecules (i.e., modification molecules). For lactate and glucose, the CLC microdroplets may be functionalized with thiol modifications, while for urea, the CLC microdroplets may be functionalized with carboxylic acid, which may then respond to the oxidation product of urea. The hydrogels may also be blended with different corresponding enzymes. The working principle of the CLC microlaser is shown in FIG. 7B. FIG. 7B shows a schematic illustrating the sensing mechanism of the modified cholesteric liquid crystal (CLC) droplets for generating whispering galley modes according to various embodiments. The analytes or metabolites in the perspiration/sweat may react with the enzyme groups in the hydrogel to release certain chemicals, which may further react with the modified CLC microdroplets, leading to protonation and deprotonation of the modification molecules and the alignment of liquid crystal molecules. With the change of orientation of liquid crystal molecules, the lasing signal will also change owing to the fluctuation of the refractive index, indicating the concentration of the metabolites in sweat. FIG. 7C shows captured photoluminescence images of the luminescent modified cholesteric liquid crystal (CLC) droplets doped with different dyes and embedded in the hydrogel film according to various embodiments. As shown in FIG. 7C, the modified CLC microdroplets doped with different dyes may be evenly distributed in the hydrogel film. 4′-Pentyl-4-biphenylcarbonitrile (5CB) a and chiral dopant(S)-4-Cyano-4′-(2-methylbutyl) biphenyl may be used to form the CLC droplets. The droplet sizes may be similar for optimized sensing performance. The dyes used may be 1,3,5,7-tetramethyl-8-phenyl-4,4-difluoroboradiazaindacene (Bodipy-1), Nile Red, and 2,8-diethyl-1,3,5,7-tetramethyl-9-phenylbipyrromethene difluoroborate (Bodipy-2).

The molecular structures of materials and dyes to fabricate cholesteric liquid crystal microdroplets and the absorption/photoluminescence properties of the dyes are illustrated in FIG. 7D. FIG. 7D shows the molecular structures of (a) 4′-Pentyl-4-biphenylcarbonitrile (5CB), (b) (S)-4-Cyano-4′-(2-methylbutyl) biphenyl, (c) 1,3,5,7-tetramethyl-8-phenyl-4,4-difluoroboradiazaindacene (Bodipy-1), (d) 2,8-diethyl-1,3,5,7-tetramethyl-9-phenylbipyrromethene difluoroborate (Bodipy-2), and (e) Nile red according to various embodiments. FIG. 7E shows a three-dimensional (3D) plot of intensity as a function of wavelength (in nanometer or nm) and dye type illustrating the absorption/photoluminescence properties of the dyes according to various embodiments.

### Results

### Optical Characterization of Modified CLC Microdroplets

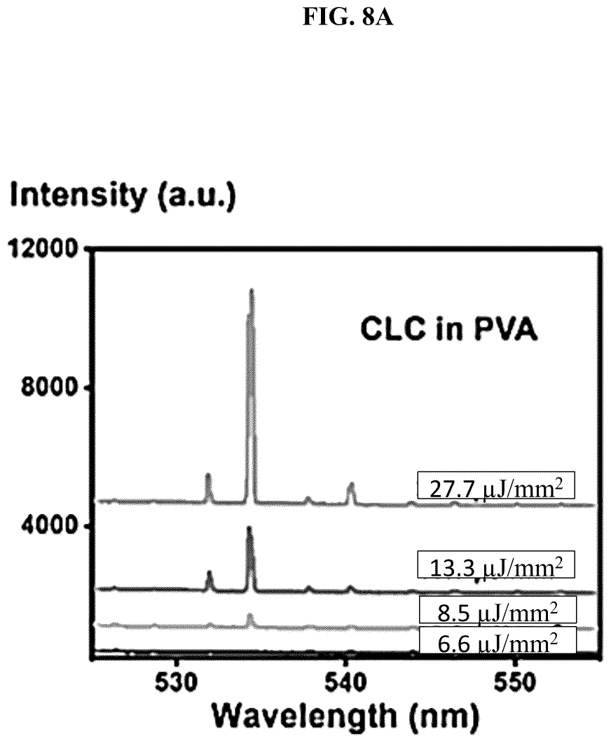

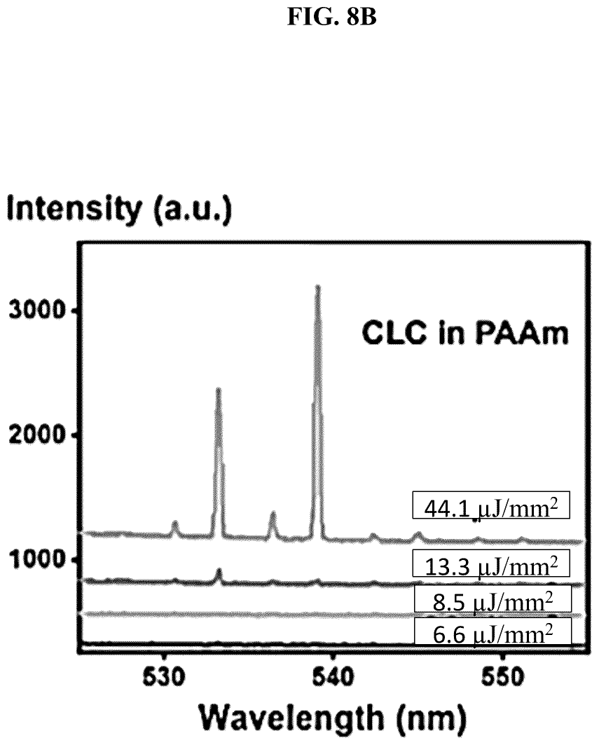

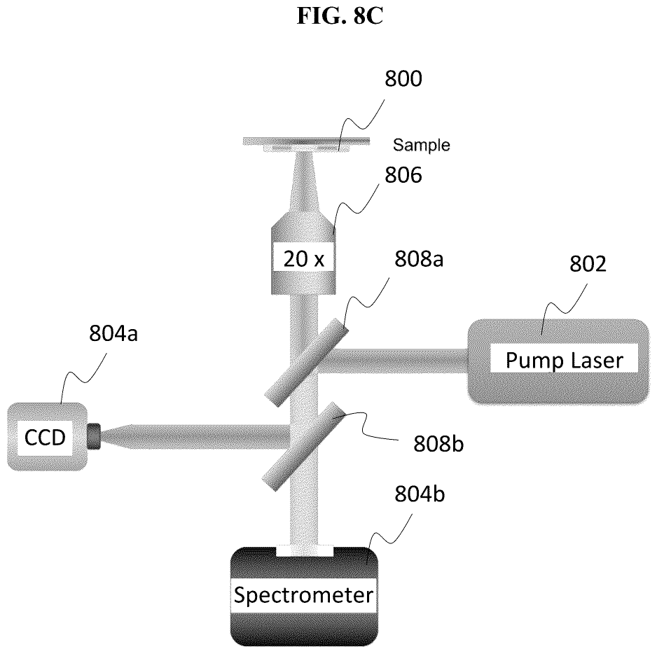

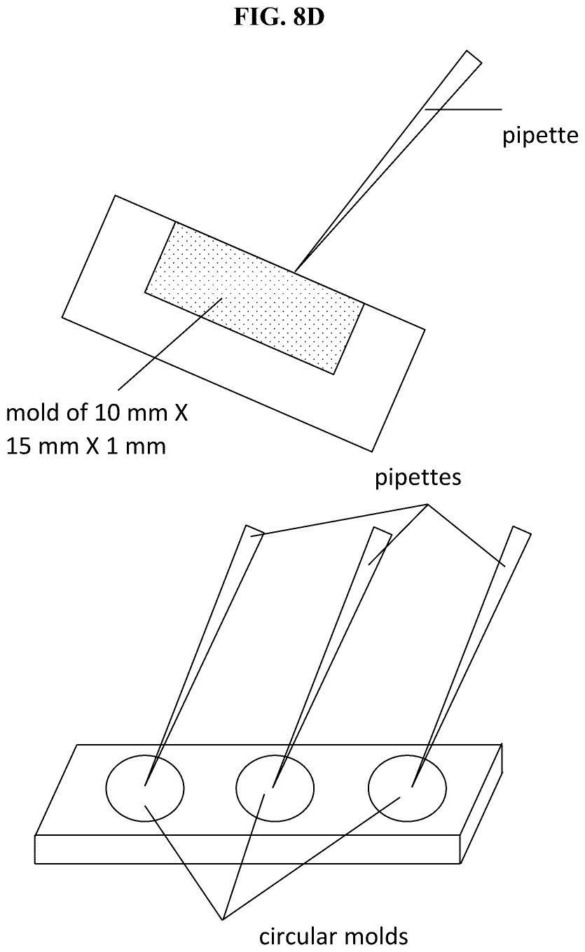

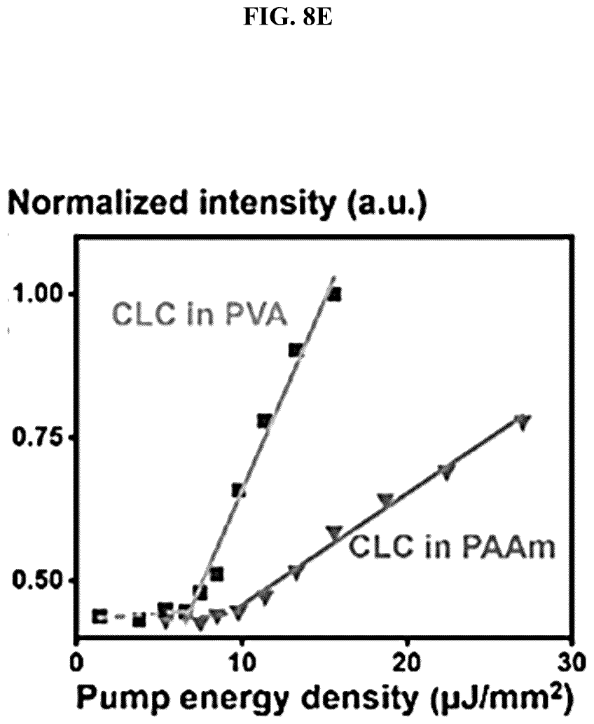

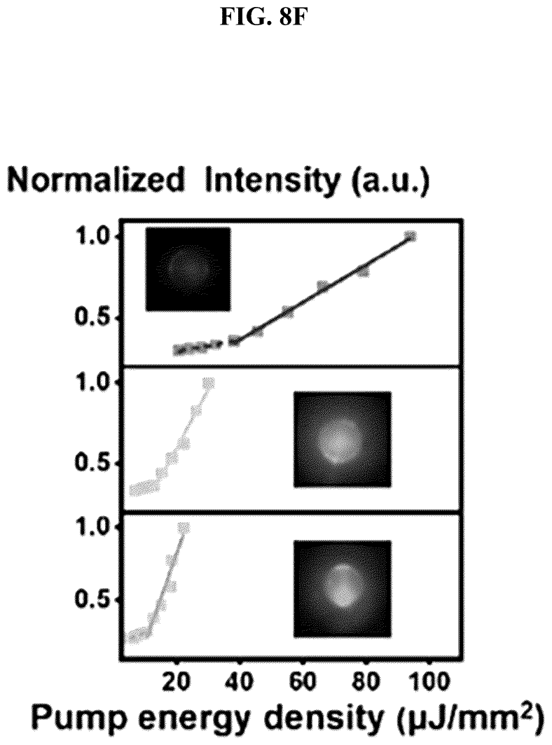

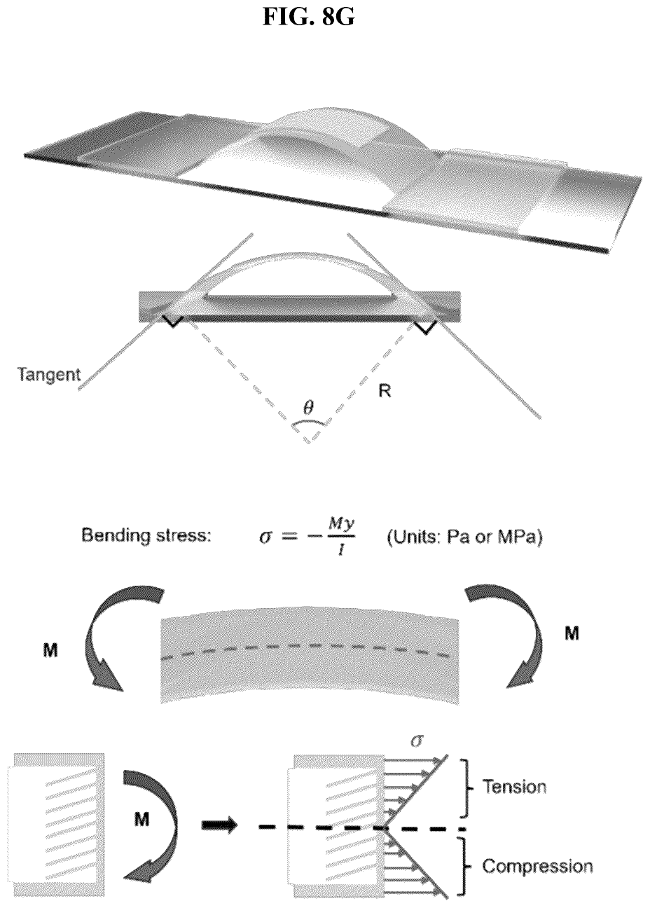

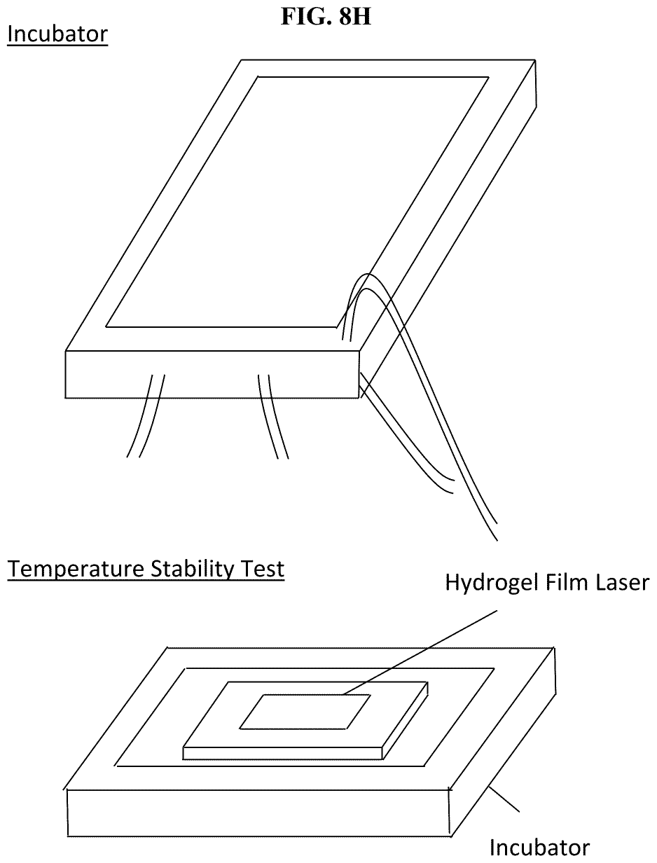

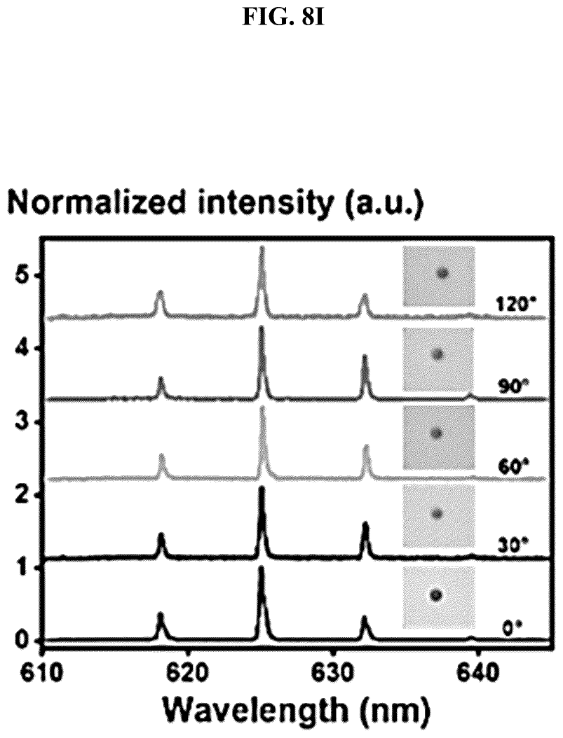

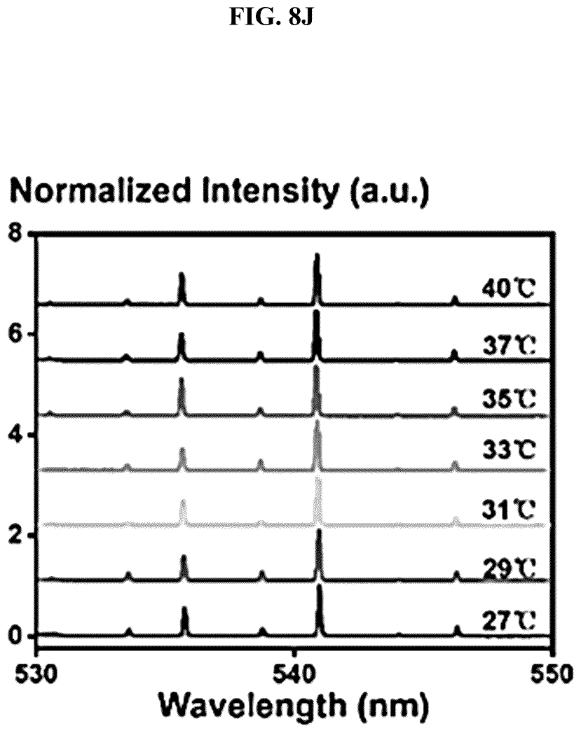

Modified CLC droplets were first prepared in a surfactant such as poly(vinyl alcohol) (PVA) solution, yielding a strong lasing emission as illustrated FIG. 8A. FIG. 8A shows a plot of intensity (in arbitrary units or a.u.) as a function of wavelength (in nanometers or nm) illustrating the lasing spectra of modified cholesteric liquid crystal (CLC) microdroplets according to various embodiments in poly(vinyl alcohol) (PVA) solution under different pump energy densities. Subsequently, the lasing performance of modified CLC microdroplets when transferred into a polyacrylamide (PAAm) hydrogel thin film material (i.e., hydrogel film) may be investigated. FIG. 8B shows a plot of intensity (in arbitrary units or a.u.) as a function of wavelength (in nanometers or nm) illustrating the lasing spectra of modified cholesteric liquid crystal (CLC) microdroplets according to various embodiments in polyacrylamide (PAAm) under different pump energy densities.

FIG. 8C shows a schematic of the optical sensing system or setup according to various embodiments. The optical sensing system or setup may include a sample including the optical sensor 800, a pump lasing subsystem (also referred to as pump laser) 802 and a detection sub-system (including a charge-coupled device (CCD) 804a and spectrometer 804b). The optical sensing system or setup may include an objective 806 configured to focus the pump laser beam onto the sample and a mirror 808a configured to direct the pump laser beam from the pump laser 802 to the objective 806. The optical sensing system or setup may also include a further mirror 808b configured to direct part of the emission beam (generated by the optical sensor 800 in response to the pump laser beam incident onto the optical sensor 800) to the CCD 804a, while allowing a remaining part of the emission beam to continue to the spectrometer 804b. FIG. 8D shows (above) a schematic illustrating fabrication of cholesteric liquid crystal (CLC) microdroplets in the polyacrylamide (PAAm) according to various embodiments in a mold of 10 mm×15 mm×1 mm; and (below) a schematic illustrating fabrication of cholesteric liquid crystal (CLC) microdroplets in the polyacrylamide (PAAm) according to various embodiments in circular molds of diameters of 8 mm.

With an increasing pump energy density, the photoluminescence intensity at ˜540 nm was amplified dramatically, manifesting the lasing action from the Bodipy-1 dye molecules. However, the lasing threshold of CLC microdroplets in PAAm hydrogel may be higher owing to the relatively higher refractive index of PAAm hydrogel as compared to PVA solution. FIG. 8E shows a plot of normalized intensity (in arbitrary units or a.u.) as a function of pump energy density (in micro-Joules per square milli-meter or mJ/mm²) illustrating the lasing threshold of dye-doped cholesteric liquid crystal (CLC) microdroplets in poly(vinyl alcohol) (PVA) and polyacrylamide (PAAm) according to various embodiments.

FIG. 8F shows a plot of normalized intensity (in arbitrary units or a.u.) as a function of pump energy density (in micro-Joules per square milli-meter or mJ/mm²) illustrating the lasing thresholds of cholesteric liquid crystal (CLC) in polyacrylamide (PAAm) with different dyes (Bodipy-1, Bodipy-2, and Nile Red) according to various embodiments, with insets showing bright whispering gallery mode (WGM) resonators lasing emissions observed at the outer boundary of the microdroplets. The lasing emissions may demonstrate a total internal reflection of the emitted light along the edge of the microcavity.

To further explore the stability of the CLC droplet resonators in PAAm hydrogel for flexible and wearable applications, bending and temperature stability tests were carried out. FIG. 8G shows (above) a schematic of the bending test for the polyacrylamide (PAAm) hydrogel film laser according to various embodiments; and (below) the bending stress distribution of the polyacrylamide (PAAm) hydrogel film according to various embodiments. The film was attached to a flexible polyimide film using glue. Subsequently, the bending test was conducted by altering the distance between two cut slides. Here R stands for bend radius and θ stands for the bend angle. For the bending stress distribution, σ is the bending stress; M is the internal bending moment at the region of interest (units: Nm); y is the perpendicular distance from the neutral axis (units: m or mm); and I is the moment of inertia about the neutral-axis for the cross-section (units: m⁴ or mm⁴). The sign “+” indicates tension, while the sign “−” indicates compression. Since the CLC microdroplets observed were at the center of the hydrogel film, the internal bending stress of the hydrogel may be near 0. This may lead to minimal stress on the CLC microdroplets and the laser signal may remain stable.

FIG. 8H shows (above) a schematic of the incubator used for the temperature stability test of the polyacrylamide (PAAm) hydrogel film laser according to various embodiments; and (below) a schematic of the temperature stability test conducted on the polyacrylamide (PAAm) hydrogel film laser according to various embodiments. To perform the temperature stability test on the PAAm hydrogel film laser, an incubator (H301-K-FRAME from Okolab) as shown in FIG. 8H may be used to maintain a consistent temperature environment. The PAAm hydrogel film was placed onto a slide and securely enclosed within the incubator. Temperature settings ranged from 27° C. to 40° C. Laser signals from identical CLC microdroplets were recorded each time the temperature within the incubator stabilized.

FIG. 8I shows a plot of normalized intensity (in arbitrary units or a.u.) as a function wavelength (in nanometers or nm) illustrating the lasing spectra of Nile Red-doped cholesteric liquid crystal (CLC) microdroplets in polyacrylamide (PAAm) with different bending angles (0° to 120°), with the insets showing morphology of the cholesteric liquid crystal (CLC) microdroplets at different bending angles. As shown in FIG. 8I, the size and shape of the CLC microdroplets (inset) remained unchanged when the hydrogel film was bent from 0° to 120° (see bending test as illustrated in FIG. 8G), leading to stable lasing performance. This may be due to the little pressure applied on the CLC microdroplets when droplets are located at the center of the hydrogel film.

FIG. 8J shows a plot of normalized intensity (in arbitrary units or a.u.) as a function wavelength (in nanometers or nm) illustrating the lasing spectra of Bodipy-1-doped cholesteric liquid crystal (CLC) microdroplets in polyacrylamide (PAAm) under different environmental temperatures, from 27° C. to 40° C. The presence of the water in the hydrogel may retain the temperature at a certain value despite the temperature change at the outer environment (as tested using the temperature stability test shown in FIG. 8H). The observation time related to FIG. 8J is only 10 minutes. In addition, unlike the CLC microdroplets in a liquid environment where the pitch increases significantly with increasing temperature. The hydrogel structure around the CLC microdroplets may limit the expansion of the CLC microdroplets, which leads to a steady laser performance during the temperature change from 27° C. to 40° C.

### Lactate Sensing With Modified CLC Microdroplets in PAAm Film

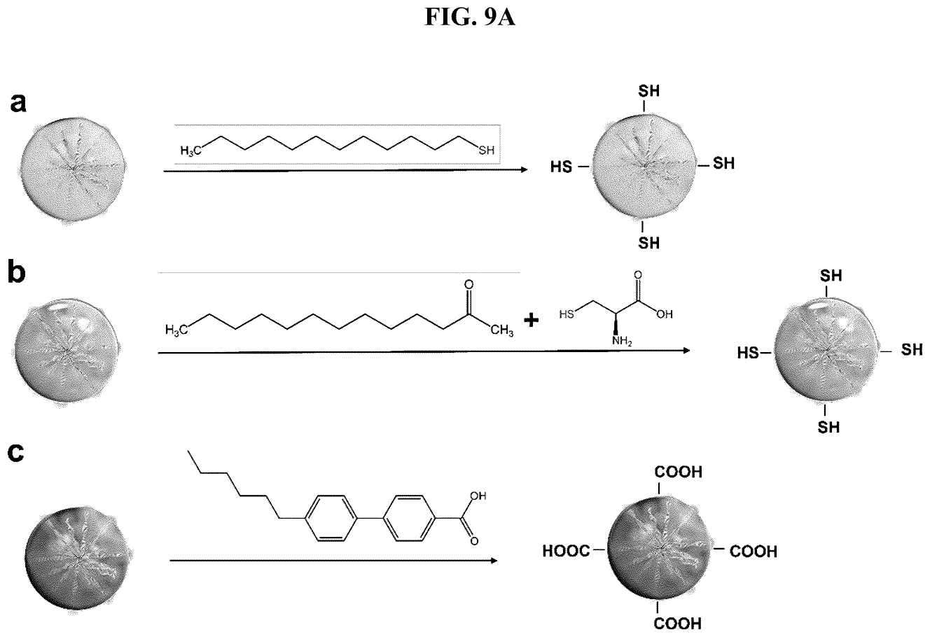

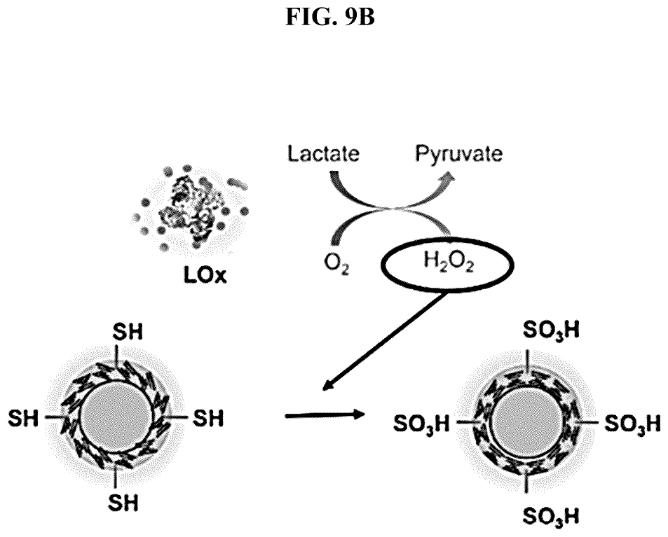

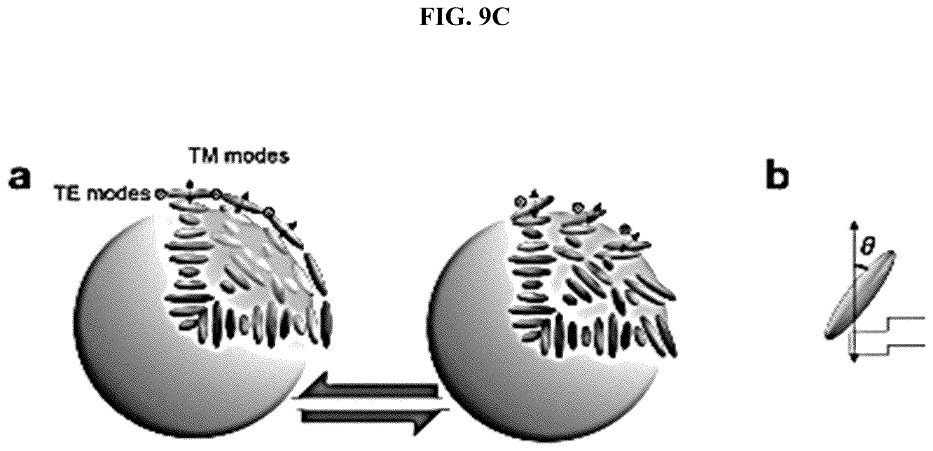

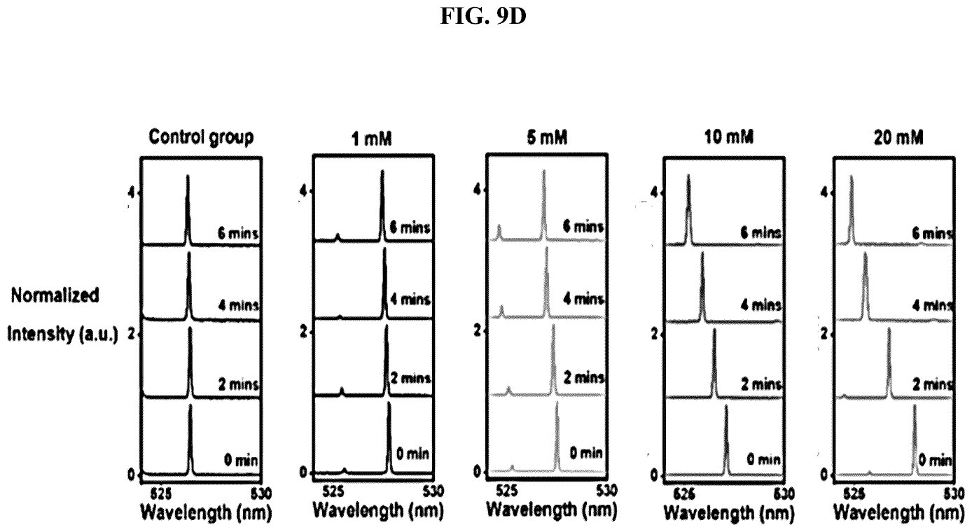

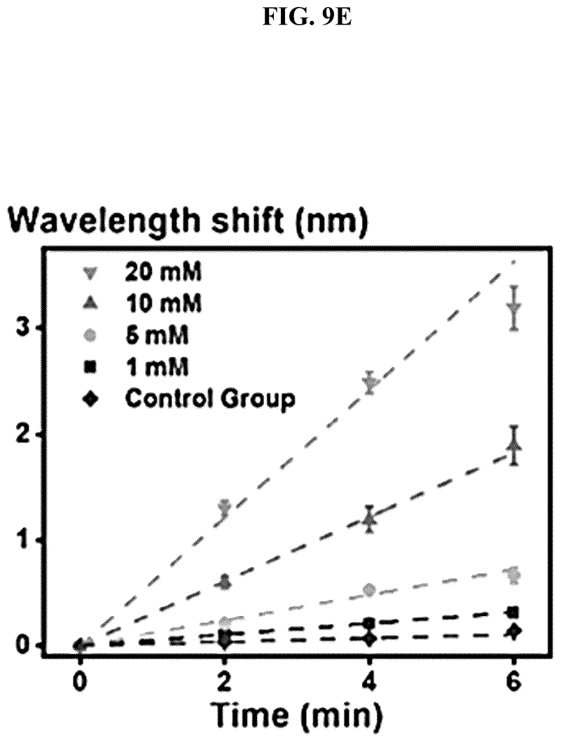

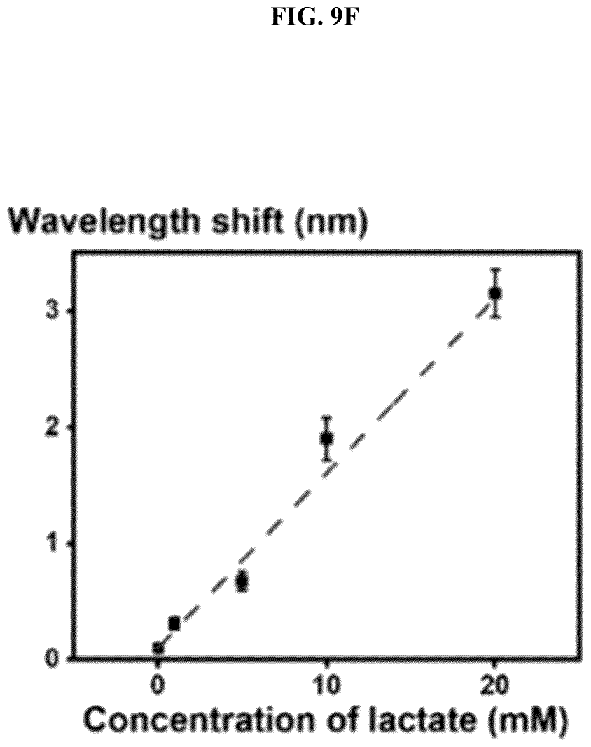

Next, the sensing capability of CLC droplet lasers encapsulated in the PAAm hydrogel thin film may be explored. Taking advantage of the orientation shift of liquid crystal molecules during the protonation and deprotonation of changed molecules, liquid crystal droplets have been employed for sensing in many applications. As such, the surface of CLC droplets can be easily modified with various molecules, allowing versatile and designable functionality. FIG. 9A shows the modification of the cholesteric liquid crystal (CLC) microdroplets for (a) lactate sensing, (b) glucose sensing and (c) urea sensing according to various embodiments. For instance, to obtain lactate sensing functionality, the hydrophilic thiol, 1-dodecanethiol, may be employed to align with the liquid crystal molecules.

FIG. 9B shows a schematic illustrating the working principle of the modified cholesteric liquid crystal (CLC) microdroplet for lactate sensing according to various embodiments. The lactate oxide (LOx), which is fixed into the PAAm hydrogel during the manufacturing of the flexible laser, may assist in oxidizing lactate molecules when lactate molecules enter the PAAm hydrogel. The oxidation of lactate may liberate pyruvate and H₂O₂ into the PAAm film, which may then oxidize the thiol (—SH) on the surface of the CLC microdroplets into sulfonyl hydroxide (—SO₃H). Since the polarity of —SO₃H is stronger than that of —SH, the process can be seen as a kind of protonation, leading to the rotation of the liquid crystal molecules. Owing to the decrease in the refractive index which TM modes sense, the lasing wavelength of microdroplets may experience a blue shift.

FIG. 9C shows whispering galley mode (WGM) polarization schematics in liquid crystal (LC) droplets illustrating (a) electric filed direction for transverse magnetic (TM) modes according to various embodiments; and (b) the birefringence of a single liquid crystal (LC) molecule. To analyze the wavelength shifts resulting from the change in the orientation of the liquid crystal molecules, there may be a need to first examine their polarization properties. There are two possible polarizations for WGMs: transverse electric (TE) polarization, where the electric field oscillates parallel to the surface of the sphere, and transverse magnetic (TM) polarization, where the electric field is perpendicular to the surface. Due to the birefringence of liquid crystal molecules, the two modes may sense different refractive indices. In the CLC droplets, as shown in FIG. 9C(a), both TE modes and TM modes may sense the lower, ordinary index (nₒ=1.52). However, as illustrated in FIG. 9C(b), when the liquid crystal molecules rotate, the TE modes may still sense the ordinary refractive index, while the TM modes may sense a combined refractive index (n) that can be calculated using the following formula Equation (1), when the rotation angle is θ″

$$n = \frac{{n}_{e} {n}_{o}}{\sqrt{{n}_{o}^{2} {sin}^{2} θ + {n}_{e}^{2} {cos}^{2} θ}} \qquad ( 1 )$$

Since nₑ>nₒ, nₒ<n<nₑ, the refractive index of the TM mode may be larger than that of the TE mode. As a result, the TM mode may be the dominant mode that is visible, owing to its larger index contrast to the outside medium. This may cause a shift in the lasing peak wavelength due to the difference between nₒ and n. To be specific, when the orientations of the liquid crystal molecules change from the left side to the right side, the lasing peaks may undergo a redshift. Otherwise, there may be a blue shift.

With the presence of lactate, significant lasing wavelength shifts were observed as the CLC droplet lasers interact with lactate molecules. FIG. 9D shows plots of normalized intensity (in arbitrary units or a,u.) as a function of wavelength (in nanometer or nm) illustrating lasing spectra of the modified cholesteric liquid crystal (CLC) microdroplets in hydrogel under different lactate concentrations according to various embodiments. The concentrations of lactate used in this experiment are 1 mM, 5 mM, 10 mM, and 20 mM. Different lactate concentrations may lead to different extents of the lasing wavelength shifts. Without the presence of lactate (i.e., the control group), the lasing peak may remain stable with less than a 0.05 nm wavelength shift. FIG. 9E shows a plot of wavelength shift (in nanometers or nm) as a function of time (in minutes or min) illustrating the wavelength shifts with increased lactate concentration after 2 minutes, 4 minutes, and 6 minutes according to various embodiments. A slight blue shift (˜0.05 nm) was found in the control group experiment (0 mM lactate), resulting from the bleaching of the Bodipy-1. FIG. 9E may reveal a linear dependence with time under various lactate concentrations (1 mM to 20 mM), indicating continuous enzyme catalysis and the release of H₂O₂. To have a fair comparison for screening analysis, the sensing results may be compared under a fixed time window of 6 minutes. FIG. 9F shows a plot of wavelength shift (in nanometers or nm) as a function of concentration of lactate (in milli-molars or mM) illustrating the wavelength shifts with increased lactate concentration under a fixed observation time (6 minutes) according to various embodiments. Error bars are obtained based on quintuplicate measurements. As illustrated in FIG. 9F, a wavelength shift of 0.31 nm, 0.67 nm, 1.89 nm, and 3.19 nm was acquired after applying 1 mM, 5 mM, 10 mM, and 20 mM lactate for 6 minutes, respectively. This range was selected according to human physiological conditions.

Glucose and Urea Sensing with Modified CLC Microdroplets in PAAm Film and Selectivity Examination

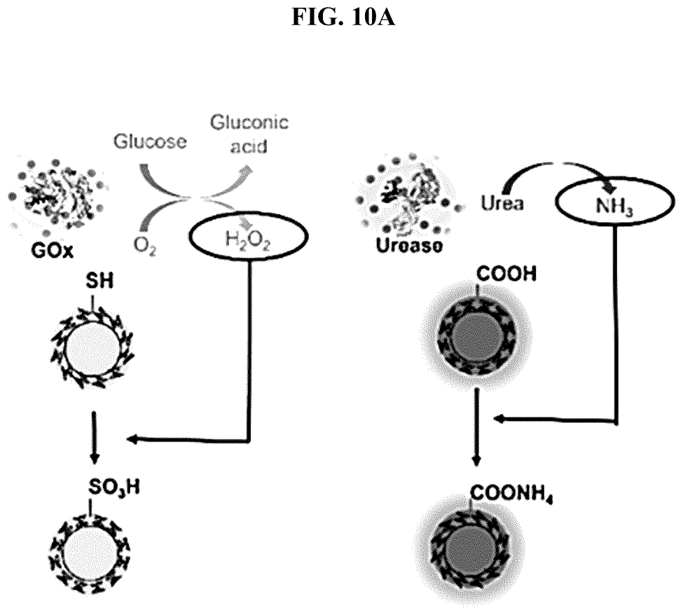

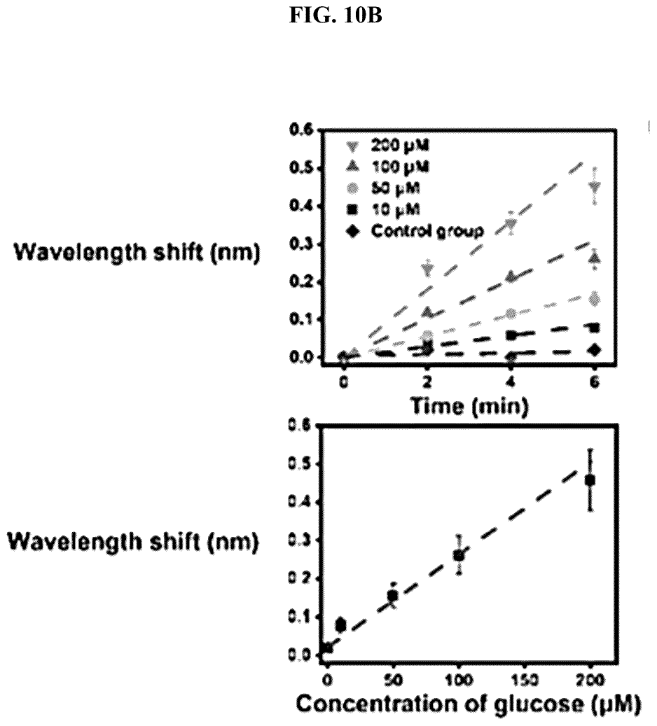

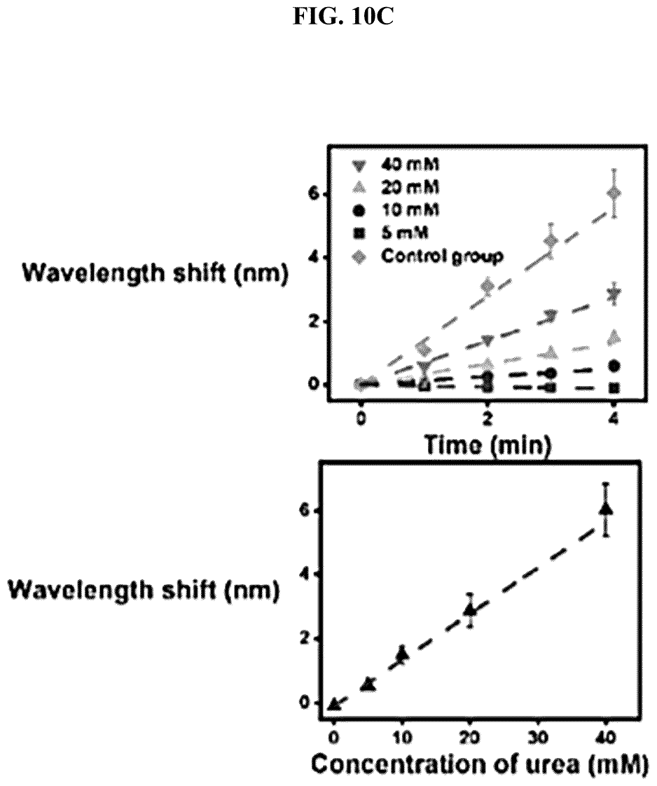

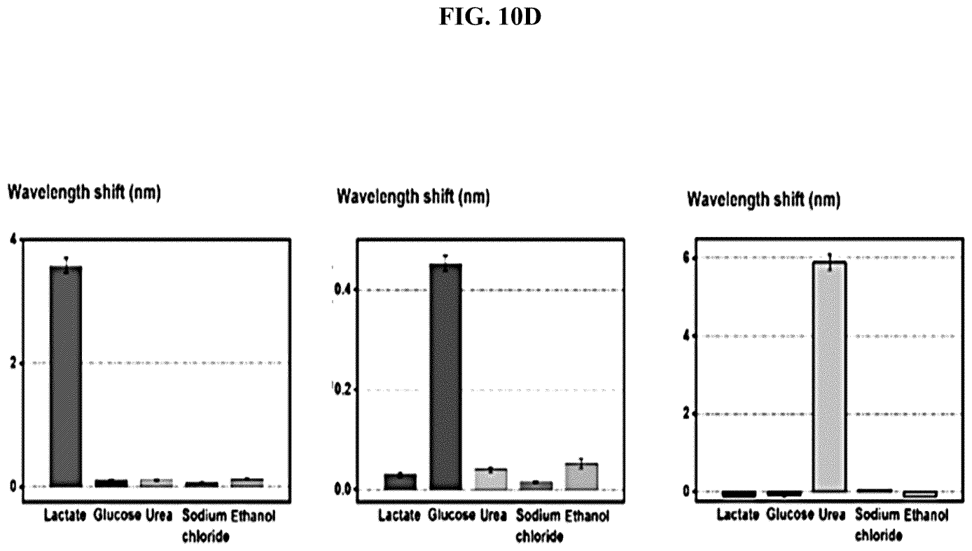

Besides detecting lactate, we next explored the possibility of using CLC droplet lasers to detect glucose and urea in the hydrogel environment. For glucose detection, a similar principle was employed owing to the generation of H₂O₂ during the reaction of glucose oxidization when glucose encounters glucose oxidase (GOx). However, the modification of CLC microdroplet lasers was adjusted to fulfill the requirements for lower glucose concentration in sweat, as shown in FIG. 9A. For urea detection, the PAAm hydrogel was blended with urease, while the carboxylic acid group (—COOH) was modified in order to respond to NH₃ (the oxidation product of urea). FIG. 10A shows a schematic illustrating the working principle of the modified cholesteric liquid crystal (CLC) microdroplet for glucose and urea sensing according to various embodiments. Note that the reaction between NH₃ and —COOH can be seen as a process of deprotonation, which may lead to a reverse rotation of liquid crystal molecules. As a result, a red shift was discovered during the urea sensing experiment in this study.

Glucose and urea were then applied to CLC microlasers. The applied concentrations were selected according to the physiological conditions in human sweat (glucose: 10 μM, 50 μM, 100 μM, 200 μM; urea: 5 mM, 10 mM, 20 mM, 40 mM). FIG. 10B shows (above) a plot of wavelength shift (in nanometers or nm) as a function of time (in minutes or min) illustrating the lasing spectra of the modified cholesteric liquid crystal (CLC) microdroplet in the hydrogel under different glucose concentrations according to various embodiments; and (below) a plot of wavelength shift (in nanometers or nm) as a function of glucose concentration (in micro-molars or μM) illustrating the wavelength shift of the lasing spectra of the modified cholesteric liquid crystal (CLC) microdroplet in the hydrogel under different glucose concentrations according to various embodiments.

FIG. 10C shows (above) a plot of wavelength shift (in nanometers or nm) as a function of time (in minutes or min) illustrating the lasing spectra of the modified cholesteric liquid crystal (CLC) microdroplet in the hydrogel under different urea concentrations according to various embodiments; and (below) a plot of wavelength shift (in nanometers or nm) as a function of urea concentration (in micro-molars or μM) illustrating the wavelength shift of the lasing spectra of the modified cholesteric liquid crystal (CLC) microdroplet in the hydrogel under different urea concentrations according to various embodiments.

FIGS. 10B-10C show a strong linear relationship between wavelength shift and different reaction times by applying different glucose and urea concentrations. Under a fixed observation time, a wavelength shift of 0.09 nm, 0.15 nm, 0.26 nm, and 0.47 nm was recorded when adding 10 μM, 50 μM, 100 μM, and 200 μM glucose solution; a wavelength shift of 0.54 nm, 1.5 nm, 2.87 nm, and 6.03 nm was recorded when adding 5 mM, 10 mM, 20 mM, and 40 mM urea solution.

Next, the selectivity of CLC microdroplets in the PAAm thin film was analyzed, as illustrated in FIG. 10D. To conduct the selectivity experiment, lactate, glucose, and urea solutions with concentrations similar to human sweat condition were prepared. Additionally, sodium chloride solution and ethanol mixture were also prepared as both are significant components in sweat. FIG. 10D shows plots of wavelength shift (in nanometers or nm) as a function of different solutions/mixtures illustrating selectivity of the optical sensor according to various embodiments. Error bars are obtained based on quintuplicate measurements. As presented in FIG. 10D, a significant lasing wavelength shift was only observed for the CLC microdroplets modified to detect lactate, glucose, or urea, respectively. Results obtained may demonstrate that the modified CLC microlasers (i.e., optical sensors) according to various embodiments possess a good selectivity with only minimum effect on the wavelength shift. Both sodium chloride and ethanol at the concentration in human sweat showed a negligible impact on the wavelength shift as well.

### Demonstration of Modified CLC Microdroplets in PAAm Hydrogel Film for Sweat Sensing

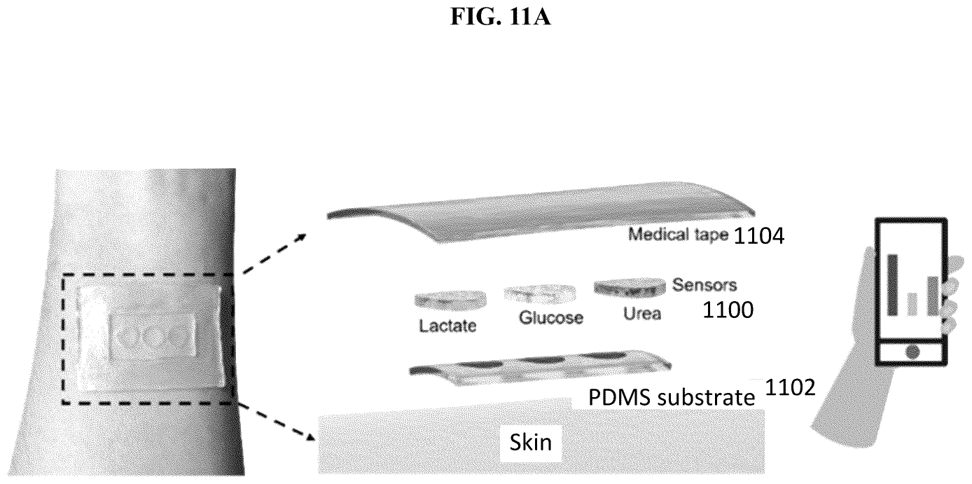

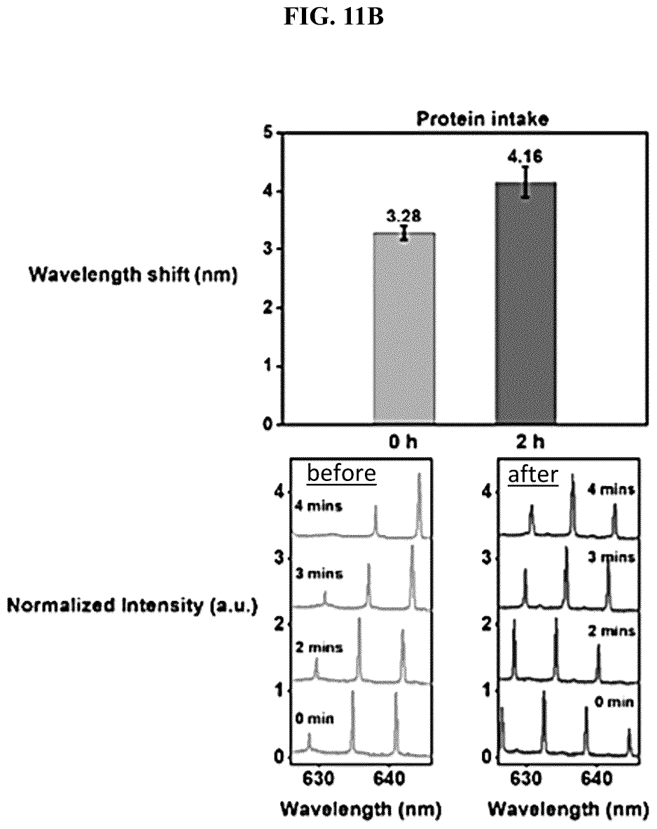

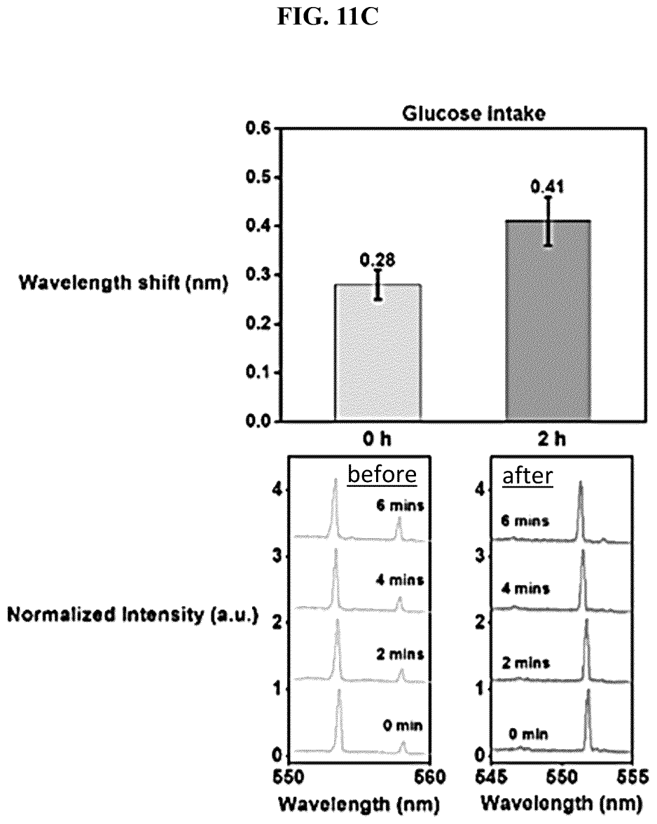

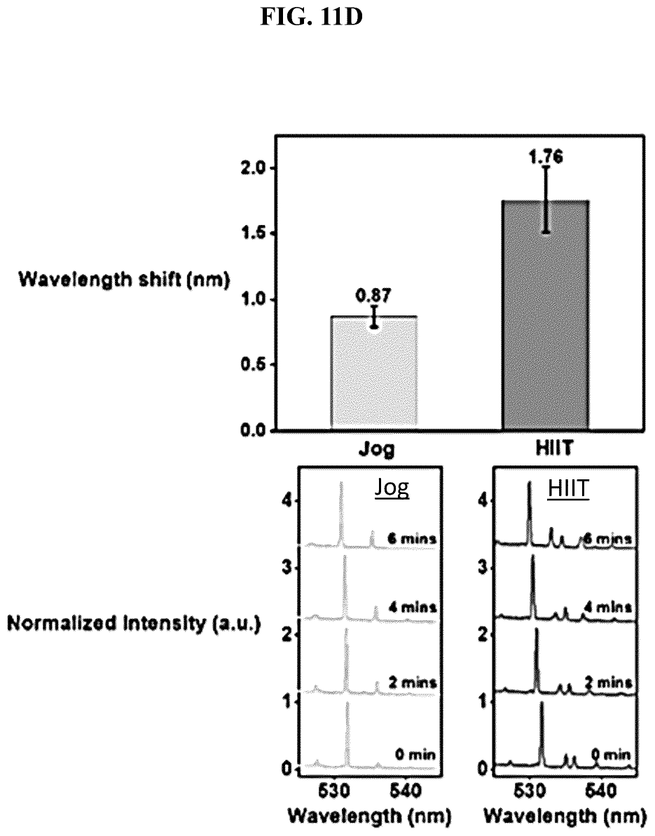

FIG. 11A shows a schematic illustrating the wearable sensing device according to various embodiments adhered to human skin for direct sweat sensing. The integrated device may include three primary pieces: lasing sensor sections 1100, a polydimethylsiloxane (PDMS) substrate 1102, and a medical tape 1104. The lasing sensor sections may be made of three different optical sensors (i.e., three different types of modified CLC microdroplets in hydrogel film). To detect three different metabolites, CLC microdroplets with three different lasing emission wavelengths were fabricated (doping liquid crystal with different gain/dyes: Bodipy-1, Bodipy-2, and Nile Red). The PDMS substrate 1102 may serve as a mold for creating the sensor components. The medical tap 1104 may be used to secure the entire apparatus to the user's skin. This wearable design may hold great potential for future use with mobile phones for wireless connectivity and the immediate transmission of health information.

Three sensors were tested independently using different methods to create proper conditions. In the context of urea sensing testing, this research endeavor aimed to investigate the wavelength shift of the modified CLC microdroplets in polyacrylamide (PAAm) hydrogel containing urease, both before and after protein intake for 2 hours. FIG. 11B shows (above) a plot of wavelength shift (in nanometers or nm) as a function of time (in hours or h) illustrating the wavelength shift of emission light emitted by the wearable sensing device according to various embodiments before and after protein intake for 2 hours; and (below) plots of normalized intensity (in arbitrary units or a.u.) as a function of wavelength (in nanometers or nm) illustrating the lasing spectra shown by the wearable sensing device according to various embodiments before and after protein intake for 2 hours. As depicted in FIG. 11B, the average wavelength shift within 6 min after protein consumption for 2 hours was found to be 4.16 nm, which is significantly higher than the preconsumption value of 3.28 nm, suggesting a higher concentration of urea in sweat. This observation is consistent with the rise in urea levels in sweat after protein consumption. Similarly, a comparable approach was employed for glucose sensing, by comparing the wavelength shift before and after consuming sugar candies for 2 h. FIG. 11C shows (above) a plot of wavelength shift (in nanometers or nm) as a function of time (in hours or h) illustrating the wavelength shift of emission light emitted by the wearable sensing device according to various embodiments before and after glucose intake for 2 hours; and (below) plots of normalized intensity (in arbitrary units or a.u.) as a function of wavelength (in nanometers or nm) illustrating the lasing spectra shown by the wearable sensing device according to various embodiments before and after glucose intake for 2 hours. As demonstrated in FIG. 11C, a change in the average wavelength shift was observed, with an increase from 0.28 to nm to 0.41 nm, indicating a higher concentration of glucose in sweat. This phenomenon may be in accordance with the rise in glucose levels in sweat after consuming sweets. With respect to lactate sensing in sweat, testing was conducted after jogging and high-intensity interval training (HIIT) since lactate secretion is closely related to exercise intensity, where higher intensity results in more lactate secretion. FIG. 11D shows (above) a plot of wavelength shift (in nanometers or nm) as a function of activity illustrating the wavelength shift of emission light emitted by the wearable sensing device according to various embodiments after jogging for 10 minutes and after high-intensity interval training (HIIT) for 10 minutes; and (below) plots of normalized intensity (in arbitrary units or a.u.) as a function of wavelength (in nanometers or nm) illustrating the lasing spectra shown by the wearable sensing device according to various embodiments after jogging for 10 minutes and after high-intensity interval training (HIIT) for 10 minutes. FIG. 11D illustrates the results of the two experiments, in which the wavelength shifts of the modified CLC microdroplets resonators after a 10 min HIIT were much higher than those after a 10 min jog (1.76 nm to 0.87 nm). The three experiments may demonstrate that the device according to various embodiments is capable of human sweat testing.

Various embodiments may relate to flexible and multifunctional microlasers that can be tailored to detect various biosignals in human sweat. By embedding modified CLC microdroplets within a PAAm hydrogel film, both flexibility and physiological sensing capabilities may be achieved on human skin, including lactate, glucose, and urea. Remarkable levels of sensitivity and minimal limits of detection may be attained across these three analytes. For lactate detection, a sensitivity of 0.15 nm/mM with a detection limit as low as 0.36 mM may be achieved. In the case of glucose, sensitivity of 0.002 nm/μM, with a low detection limit of 1.55 μM may achieved. Furthermore, for urea detection, the sensitivity may be 0.14 nm/mM, and the detection limit may be 0.31 mM. According to previous clinical studies and reports, the normal ranges for human lactate, glucose, and urea are around 5 mM-20 mM, 10 μM-200 μM, and 2 mM-10 mM respectively. Various embodiments may be able to fulfill the required dynamic range. Various embodiments may be envisioned to be applied to daily health monitoring, due to associated low costs and being disposable. Various embodiments may be used to detect any desired target metabolites by simply modifying the CLC microdroplets.

Various embodiments may relate to an emitting plaster (bandage) for multiplexed detection through a noninvasive wearable laser device. The emitting plaster (bandage) can quickly detect metabolites in 2 minutes through sweat secreted on human skin. Various embodiments may be formed by embedding tiny optical sensors in a hydrogel patch. The bandage may use laser light emitted from the bandage to identify the tiny fluctuations of glucose level in sweat and can offer a record low limit of detection. In addition, various embodiments can detect multiple metabolites at the same time to help monitor our health conditions more precisely.

By embedding modified CLC microdroplets within a PAAm hydrogel film, both flexibility and physiological sensing capabilities on human skin (including lactate, glucose, and urea) may be achieved. Various embodiments may achieve remarkable levels of sensitivity and minimal limits of lactate, glucose, and urea detection. Various embodiments may fulfill the required dynamic range, and may be applicable to daily health monitoring, as it is low-cost and disposable. Furthermore, various embodiments can be used to detect any desired target metabolites by simply modifying the CLC microdroplets. Various embodiments may also be very versatile. By altering the components of the droplets or the hydrogel film itself, the structure of microdroplets in the hydrogel film can be adjusted to any suitable lasing wavelengths.

It may be envisioned that the uniformity and distribution of the CLC microdroplet sensors can be improved by implementing advanced microfluidic systems or imprinted technology to form an array-like sensor with a larger sensing area. Also, as mentioned above, by modification of the CLC droplets, the range of detectable biomolecules can be expanded, such as drugs secreted in sweat, pathological chemicals, and others. The abilities of this structure of microdroplets in the hydrogel film can be enhanced by altering the components of the droplets or the hydrogel substrate itself. Additionally, by integrating the microlaser with miniaturized laser diodes and even on-chip spectrometers, a flexible and wearable photonic chip can be created for human health monitoring.

Flexible wearable devices may have great potential to integrate with human interfaces, and may provide real-time monitoring of key physiological information and more clinical-relevant bioinformation. The functionality of micro-resonators can be simply tended to detect a wide range of analytes in human sweat. Furthermore, the narrow linewidth of laser emissions may make multiplexing more achievable.

### Optical System Setup

A microscope system (Nikon Ni2) with 20×0.3 numerical aperture (NA) objective was used to pump the microdroplets resonator and collect light. The optical pumping was performed using a pulsed nanosecond laser (EKSPLA NT230) integrated with an optical parametric oscillator, with a repetition rate of 50 Hz and a pulse duration of 5 ns. For Bodipy-1 and Bodipy-2, the excitation wavelength was set to 488 nm, while for Nile red, it was set to 532 nm. The beam diameter at the objective focal plane was approximately 20 μm. The emission laser from the microspheres was split by a beam splitter and directed to a spectrometer (Andor Kymera 328i) and a complementary metal oxide semiconductor (CMOS) camera (Andor Zyla 5.5) for spectrum and image acquisition, respectively. A color charge-coupled device (CCD) camera (DS-Fi3, Nikon) was mounted on the microscope to measure the color fluorescence images of microdroplets.

### Fabrication of Modified CLC Microdroplets

Cholesteric liquid crystal (CLC) mixture was obtained by mixing 4′-Pentyl-4-biphenylcarbonitrile (Sigma, 328510) with the chiral dopant(S)-4-Cyano-4′-(2-methyl butyl) biphenyl (Tokyo Chemical Industry, C2913) in a concentration of 15 wt %. Then, the modified CLC microdroplets are fabricated by sonification in polyvinyl alcohol (PVA) solution and selected by a syringe filter, whose diameters are around 12 μm˜13 μm.

For lactate sensing, 10 mM Bodipy-1 (Sigma, 793728) and 0.1 wt. % 1-dodecanethiol (Sigma, 471364) were doped into the CLC mixture. Bodipy-1 was used as the gain medium for lasing and the 1-dodecanethiol served as the thiol modification. 10 μL doped CLC mixture was added to 1 mL polyvinyl alcohol (PVA) solution (1 wt. %). The final mixture was treated with 1 min sonication to generate LC droplets. The obtained modified CLC microdroplets solution was filtered by using a syringe filter with a pore size of 15 μm.

For glucose sensing, a 10 mM Bodipy-2 (Sigma, 795526) and 2.5 wt % lauroyl chloride (Tokyo Chemical Industry, D0972) were doped into the CLC mixture. Bodipy-2 served as the gain medium for lasing while lauroyl chloride acted as the tailoring agent for thiol modification. Subsequently, 10 μL of the freshly prepared doped CLC mixture was added to 1 mL of polyvinyl alcohol (PVA) solution (1 wt. %). The final mixture was sonicated for 1 minute to generate LC droplets, and the resulting LC droplets solution was filtered using a syringe filter with a 15 μm pore size. A solution of L-cysteine with a concentration of 1 mM was freshly prepared by dissolving L-cysteine powder (Sigma, 168149) into phosphate-buffered saline solution (pH=8.0, containing 1 wt. % PVA). After centrifugation, the LC droplets were dispersed into the L-cysteine solution and incubated for 30 minutes. Finally, the modified CLC microdroplets were centrifuged and then dispersed into 1 wt. % PVA for use.

For urea sensing, 15 mM Nile Red (Sigma, 72485) and 0.25 wt % of 4′-n-hexyl biphenyl-4-carboxylic acid (Alfa Aesar, B21900.03) was doped into the liquid crystal. 10 μL doped CLC mixture was added to 1 mL polyvinyl alcohol (PVA) solution (1 wt. %). The final mixture was treated with 1 min sonication to generate modified CLC microdroplets. The obtained modified CLC microdroplets solution was filtered by using a syringe filter with 15 μm pore size.

Various embodiments including other components (e.g., other photoluminescent dyes and/or other modification molecules) may also be formed using similar steps as described above.

### Fabrication of The Modified CLC Microdroplets in PAAm Hydrogel

For PAAm hydrogel precursor solution, 100 mg acrylamide and 1 mg N,N′-methylenebis(acrylamide) (Sigma, 146072) were dissolved in 900 μL deionized (DI) water. Furthermore, 10 mg photoinitiator (2-hydroxy-4′-(2-hydroxyethoxy)-2-methypropiophenone) was added to the solution to trigger polymerization after ultraviolet (UV) curing (Panasonic #ANUJ3500). Then, the precursor solution may be modified by adding 0.1 mg lactate oxidase, 0.1 mg glucose oxidase, or 0.1 mg urease respectively for lactate, glucose, and urea sensing. All the modified CLC microdroplets solution (in 1% PVA solution) was centrifuged and then dispersed into modified PAAm hydrogel precursor solution, with specific modified CLC microdroplets corresponding to specific modified precursor solutions. The resulting solution was poured into prepared molds, and the hydrogel was fabricated after UV curing for 30 s. To remove any unreacted monomer, crosslinker, and photoinitiator, the hydrogel was soaked in DI water for 10 minutes. Two molds were utilized in this study: a cuboid mold, which was fabricated by cutting slides to a size of 10 mm*15 mm*1 mm, and a circular mold, which was fabricated by attaching PDMS to a slide with a diameter of 8 mm. The microdroplets may be randomly distributed in the hydrogel. The microdroplets lying at the middle height of the hydrogel film may be chosen for the performance test. For the sensing application, microdroplets of similar sizes may be selected to get consistent and reliable results.

Various embodiments including other components (e.g., other hydrogels and/or enzymes) may also be formed using similar steps as described above.

### Testing Experiment on Skin

In the urea and glucose sensing study, the participant was instructed to walk outdoors with plastic wrap wrapped around the arm to induce perspiration. After perspiration, the device was attached to the arm for 30 seconds to collect the sweat. The lasing testing was then performed over a period of 6 minutes. After the initial walk, participants were given a standardized protein drink containing 30 g of protein for the urea sensing test, and two energy bars with a total of 54 g of sugar for the glucose sensing test. After a 2-hour period, the same testing process was repeated.

For the lactate sensing test, the participant was instructed to jog for 10 minutes with plastic wrap wrapped around their arm. After jogging, the device was attached to the arm for 30 seconds to collect the sweat. The lasing testing was then performed over a period of 6 minutes. The participant was given a 30-minute rest period before performing a 10-minute high-intensity interval training (HIIT) exercise. Finally, the same testing process was repeated.

1. An optical sensor for detecting an analyte, the optical sensor comprising:
a hydrogel film;
one or more cholesteric liquid crystal (CLC) droplets mixed with the hydrogel film, each of the one or more cholesteric liquid crystal (CLC) droplets comprising a plurality of liquid crystal molecules; and
modification molecules attached to the one or more cholesteric liquid crystal (CLC) droplets;
wherein the one or more cholesteric liquid crystal (CLC) droplets are doped with a photoluminescent dye;
wherein the one or more cholesteric liquid crystals (CLC) droplets are configured to generate an emission beam in response to a pump laser beam incident onto the one or more cholesteric liquid crystals (CLC) droplets; and
wherein the one or more cholesteric liquid crystal (CLC) droplets are configured as whispering gallery mode (WGM) resonators such that a wavelength of the emission beam is shifted in response to the modification molecules coming in contact with a chemical derived from the analyte, thereby detecting the analyte.

2. The optical sensor according to claim 1,
wherein the hydrogel film comprises an enzyme configured to react with the analyte to form the chemical.

3. The optical sensor according to claim 1,
wherein the shift in the wavelength of the emission beam is due to a change in orientation of the plurality of liquid crystal molecules as a result of a reaction between the chemical and the modification molecules.

4. The optical sensor according to claim 1,
wherein the modification molecules are thiol molecules or carboxylic acid molecules.

5. The optical sensor according to claim 1,
wherein the photoluminescent dye is selected from a group consisting of Bodipy-1, Bodipy-2, and Nile Red.

6. The optical sensor according to claim 1,
wherein the hydrogel film comprises polyacrylamide (PAAm).

7. The optical sensor according to claim 1,
wherein the analyte is lactate, glucose or urea.

8. An optical sensing system comprising:
one or more optical sensors, each of the one or more optical sensors comprising:
a hydrogel film;
one or more cholesteric liquid crystal (CLC) droplets mixed with the hydrogel film, each of the one or more cholesteric liquid crystal (CLC) droplets comprising a plurality of liquid crystal molecules; and
modification molecules attached to the one or more cholesteric liquid crystal (CLC) droplets;
wherein the one or more cholesteric liquid crystal (CLC) droplets are doped with a photoluminescent dye;
wherein the one or more cholesteric liquid crystals (CLC) droplets are configured to generate an emission beam in response to a pump laser beam incident onto the one or more cholesteric liquid crystals (CLC) droplets;
wherein the one or more cholesteric liquid crystal (CLC) droplets are configured as whispering gallery mode (WGM) resonators such that a wavelength of the emission beam is shifted in response to the modification molecules coming in contact with a chemical derived from the analyte, thereby detecting the analyte; and
wherein the optical sensing system further comprises:
a pump lasing sub-system configured to provide the pump laser beam; and
a detection sub-system configured to detect the emission beam.

9. A wearable analyte sensing device comprising:
one or more optical sensors,
each of the one or more optical sensors comprising:
a hydrogel film;
one or more cholesteric liquid crystal (CLC) droplets mixed with the hydrogel film, each of the one or more cholesteric liquid crystal (CLC) droplets comprising a plurality of liquid crystal molecules; and
modification molecules attached to the one or more cholesteric liquid crystal (CLC) droplets;
wherein the one or more cholesteric liquid crystal (CLC) droplets are doped with a photoluminescent dye;
wherein the one or more cholesteric liquid crystals (CLC) droplets are configured to generate an emission beam in response to a pump laser beam incident onto the one or more cholesteric liquid crystals (CLC) droplets;
wherein the one or more cholesteric liquid crystal (CLC) droplets are configured as whispering gallery mode (WGM) resonators such that a wavelength of the emission beam is shifted in response to the modification molecules coming in contact with a chemical derived from the analyte, thereby detecting the analyte; and
wherein the wearable analyte sensing device further comprises:
a substrate holding the one or more optical sensors; and
an adhesion layer for adhering the wearable analyte sensing device to an user;
wherein the one or more optical sensors are between the substrate and the adhesion layer.

10. The wearable analyte sensing device according to claim 9,
wherein the adhesion layer is a medical tape.

11. A method of forming an optical sensor for detecting an analyte, the method comprising:
forming one or more cholesteric liquid crystal (CLC) droplets, each of the one or more cholesteric liquid crystal (CLC) droplets comprising a plurality of liquid crystal molecules;
attaching modification molecules to the one or more cholesteric liquid crystal (CLC) droplets;
doping the one or more cholesteric liquid crystal (CLC) droplets with a photoluminescent dye; and
mixing the one or more cholesteric liquid crystal (CLC) droplets with the hydrogel film;
wherein the one or more cholesteric liquid crystals (CLC) droplets are configured to generate an emission beam in response to a pump laser beam incident onto the one or more cholesteric liquid crystals (CLC) droplets; and
wherein the one or more cholesteric liquid crystal (CLC) droplets are configured as whispering gallery mode (WGM) resonators such that a wavelength of the emission beam is shifted in response to the modification molecules coming in contact with a chemical derived from the analyte, thereby detecting the analyte.

12. The method according to claim 11,
wherein the one or more cholesteric liquid crystal (CLC) droplets are doped with the photoluminescent dye and attached to the modification molecules, before mixing the one or more cholesteric liquid crystal (CLC) droplets doped with the photoluminescent dye and functionalized with the modification molecules with the hydrogel film.

13. The method according to claim 12,
wherein a cholesteric liquid crystal (CLC) mixture is mixed with the photoluminescent dye and the modification molecules to form the one or more cholesteric liquid crystal (CLC) droplets doped with the photoluminescent dye and functionalized with the modification molecules.

14. The method according to claim 11,
wherein the hydrogel film comprises an enzyme configured to react with the analyte to form the chemical.

15. The method according to claim 11,
wherein the hydrogel film is formed by mixing a hydrogel precursor solution with a photoinitiator to form a resultant mixture; and providing ultraviolet (UV) light to the resultant mixture.

16. The method according to claim 11,
wherein the modification molecules are thiol molecules or carboxylic acid molecules.

17. The method according to claim 11,
wherein the photoluminescent dye is selected from a group consisting of Bodipy-1, Bodipy-2, and Nile Red.

18. The method according to claim 11,
wherein the hydrogel film comprises polyacrylamide (PAAm).

19. A method of forming an optical sensing system, the method comprising:
forming one or more optical sensors, each of the one or more optical sensors comprising:
a hydrogel film;
one or more cholesteric liquid crystal (CLC) droplets mixed with the hydrogel film, each of the one or more cholesteric liquid crystal (CLC) droplets comprising a plurality of liquid crystal molecules; and
modification molecules attached to the one or more cholesteric liquid crystal (CLC) droplets;
wherein the one or more cholesteric liquid crystal (CLC) droplets are doped with a photoluminescent dye;
wherein the one or more cholesteric liquid crystals (CLC) droplets are configured to generate an emission beam in response to a pump laser beam incident onto the one or more cholesteric liquid crystals (CLC) droplets;
wherein the one or more cholesteric liquid crystal (CLC) droplets are configured as whispering gallery mode (WGM) resonators such that a wavelength of the emission beam is shifted in response to the modification molecules coming in contact with a chemical derived from the analyte, thereby detecting the analyte; and
wherein the method further comprises:
providing a pump lasing sub-system configured to provide the pump laser beam; and
providing a detection sub-system configured to detect the emission beam.

20. A method of forming a wearable analyte sensing device, the method comprising:
forming one or more optical sensors, each of the one or more optical sensors comprising:
a hydrogel film;
one or more cholesteric liquid crystal (CLC) droplets mixed with the hydrogel film, each of the one or more cholesteric liquid crystal (CLC) droplets comprising a plurality of liquid crystal molecules; and
modification molecules attached to the one or more cholesteric liquid crystal (CLC) droplets;
wherein the one or more cholesteric liquid crystal (CLC) droplets are doped with a photoluminescent dye;
wherein the one or more cholesteric liquid crystals (CLC) droplets are configured to generate an emission beam in response to a pump laser beam incident onto the one or more cholesteric liquid crystals (CLC) droplets;
wherein the one or more cholesteric liquid crystal (CLC) droplets are configured as whispering gallery mode (WGM) resonators such that a wavelength of the emission beam is shifted in response to the modification molecules coming in contact with a chemical derived from the analyte, thereby detecting the analyte; and
wherein the method further comprises:
forming a substrate to hold the one or more optical sensors; and
forming an adhesion layer for adhering the wearable analyte sensing device to an user;
wherein the one or more optical sensors are between the substrate and the adhesion layer.
